(19) United States
(12) Patent Application Publication
(10) Pub. No.: US 2002/0148005 A1
(43) Pub. Date: Oct. 10, 2002

(54) Method of using DNA episomes to suppress gene expression in plants

(76) Inventors: Charles Peele, Apex, NC (US); Dominique Robertson, Cary, NC (US); Michael A. Turnage, Pittsboro, NC (US)

Correspondence Address: MYERS BIGEL SIBLEY & SAJOVEC, PO BOX 37428, RALEIGH, NC, 27627, US

(21) Appl. No.: 09/876,360

(22) Filed: Jun. 7, 2001

Related U.S. Application Data
(60) Provisional application No. 60/210,141, filed on Jun. 7, 2000.

(51) Int. Cl. A01H 5/00; C12N 15/86

(52) U.S. Cl. 800/280; 435/320.1; 435/235.1

(57) ABSTRACT

The introduction of DNA episomes into plant cells to reduce or prevent the expression of endogenous plant genes is described. Cabbage leaf curl virus vectors to provide silencing, preferably systemic silencing, of endogenous plant genes in a treated plant are described. Further provided are methods of silencing one or more plant genes, for example, to reduce unwanted gene products or for rapid screening of gene function in plants

### RELATED APPLICATION INFORMATION

This application claims the benefit of United States Provisional Application No. 60/210,141, filed Jun. 7, 2000, which is incorporated by reference herein in its entirety.

### STATEMENT OF FEDERAL SUPPORT

This invention was made with government support under United States Department of Agriculture grant number NRICGP 97-35303-4538 and Tri-Agency Training Grant number NSF BIR9420689 from the National Science Foundation, United States Department of Energy, and the United States Department of Agriculture. The United States government has certain rights to this invention.

### FIELD OF THE INVENTION

The present invention relates to the introduction of DNA episomes into plant cells to silence plant genes. More particularly, this invention relates to the use of geminivirus vectors to provide silencing of one or more endogenous genes in treated plants.

### BACKGROUND OF THE INVENTION

Gene silencing in plants typically refers to the suppression of either an endogenous gene or ectopically-integrated transgene by the introduction of a related transgene. Some examples of pathogen-derived host resistance to RNA viruses have been attributed to a gene silencing mechanism (Covey et al., Nature 386, 781 (1997); Mueller et al., Plant J. 7, 1001 (1995); Ratcliff et al., Science 276, 1558 (1997); Tanzer et al., Plant Cell 9, 1411 (1997)). Transcriptional gene silencing has been hypothesized to involve DNA/DNA pairing, DNA methylation or heterochromatinization (Kumpatia et al., Plant Physiol. 115, 361 (1997); Neuhuber, Mol. Gen. Genetics 247, 264 (1995); Jones et al., EMBO J. 17, 6385 (1998); Park et al., Plant J. 9, 183 (1996)). Repeated DNA has a tendency to undergo transcriptional silencing, which may be associated with changes in chromatin structure (Meyer, Biol. Chem. Hoppe Sayler 377, 87 (1996); Ye and Singer, Proc. Natl. Acad. Sci. USA 93, 10881 (1996)), and/or to induce certain types of post-transcriptional silencing (Stam et al., Plant J. 12, 63 (1997)). Both cytoplasmic and nuclear events have been implicated in gene silencing. Post-transcriptional gene silencing may involve the synthesis of short RNA molecules, synthesized by an RNA-dependent RNA polymerase (Cogoni and Macino, Nature 399, 166 (1999)), that anneal to homologous sense RNA and thereby provide a target for a double-stranded RNase or affect RNA abundance indirectly by interfering with translation (Baulcombe, Plant Mol. Biol. 32, 79 (1996)).

Geminiviruses are single-stranded DNA viruses that replicate through double-stranded DNA intermediates using plant DNA replication machinery. Geminiviruses replicate in the nucleus, and foreign DNA can be stably integrated into the viral genome without significantly affecting replication or movement. Tomato golden mosaic virus (TGMV) and cabbage leaf curl virus (CbLCV) are bipartite geminiviruses with genomes consisting of two circular components, A and B (FIG. 1). The A component replicates autonomously whereas the B component is dependent on the A component for replication. For TGMV, the coat protein (AR1, also known as V1) is dispensable for replication and movement in Nicotiana Benthamiana and can be replaced with up to 800 bp of foreign DNA, which is stably maintained in the viral genome (Elmer and Rogers, Nucl. Acids Res. 18, 2001 (1990)). Similarly, the CbLCV coat protein can be replaced with foreign DNA.

A plant virus may systemically infect a plant by spreading from the initially-infected cell to neighboring cells, and subsequently throughout the plant. Plant cell walls prevent the random cell-to-cell transfer of the virus, but channels (plasmodesmata) that transverse plant cell walls provide an intercellular continuum through which the virus particles or viral nucleic acids may move. Viral movement via plasmodesmata is mediated by virus encoded proteins (Citovsky et al., Bioassays 13, 373 (1991)). Additionally, movement of the virus to parts of the plant distant from the site of the initial infection can occur via companion cells and sieve elements of the phloem. However, even in systemically infected plants the distribution of the virus may not be uniform. Certain areas of the plant, even within a plant tissue or a structure, may contain higher or lower amounts of virus than neighboring areas.

### SUMMARY OF THE INVENTION

The present investigations demonstrate that episomes derived from DNA plant viruses (preferably, geminiviruses) can effect silencing of active chromosomal gene expression in plants, i.e., can produce gene silencing. Preferably, the episomal silencing vectors are localized to the nucleus of the plant cell. The silencing vector comprises one or more heterologous DNA sequences, each of which has substantial sequence similarity with an endogenous plant gene or a fragment thereof (including coding and/or non-coding sequences). The silencing of nuclear genes can be achieved by the homologous sequences carried by the DNA episome. The present invention advantageously permits silencing of gene expression in intact plants, without the need for transformation followed by regeneration of entire plants.

A first aspect of the present invention is a Cabbage Leaf Curl Virus (CbLCV) silencing vector comprising a CbLCV genomic component comprising one or more heterologous DNA sequences, where each heterologous DNA sequence is identical to, or has substantial sequence similarity to, a gene endogenous to a plant (including fragments thereof). In particular embodiments, the CbLCV genomic component is the A or the B component, or a binary vector comprising both.

A further aspect of the present invention is a vector comprising a CbLCV A component, where the DNA encoding the CbLCV coat protein has been replaced in part or in total with one or more heterologous DNA sequences, each of which is identical to, or has substantial sequence similarity to, an endogenous plant gene (including fragments thereof).

A still further aspect of the present invention is a vector comprising a CbLCV B component, wherein one or more heterologous DNA sequences, each of which is identical to or has substantial sequence similarity to an endogenous plant gene or fragment thereof, is inserted into or replaces sequences within the B component. In preferred embodiments, at least one of the sequences is inserted into or replaces sequences in the 3′ non-coding region of the BR1 and/or BL1 genes.

A further aspect of the present invention is a silencing vector comprising a CbLCV origin of replication; CbLCV sequences encoding proteins sufficient for replication of the vector in a plant cell; and one or more heterologous DNA sequences that are each identical to, or have substantial sequence similarity to, an endogenous plant gene (including fragments thereof).

A yet further aspect of the present invention is a silencing vector comprising a CbLCV origin of replication; a CbLCV BR1 and/or BL1 gene (preferably both); and one or more heterologous DNA sequences that are each identical to, or have substantial sequence similarity to, one or more endogenous plant genes (including fragments thereof).

A further aspect of the present invention is a method of silencing (preferably, systemically) the expression of a plant gene in a plant cell by inoculating the plant cell with a silencing vector as described above.

A still further aspect of the present invention is a method of screening isolated plant DNA sequences for function. The method comprises preparing a silencing vector as described above, containing one or more DNA sequences identical to, or having substantial sequence similarity to, the isolated plant DNA (including fragments thereof). A test plant is then inoculated with the silencing vector and allowed to grow for a period of time, then compared to a non-inoculated or sham inoculated control plant. Differences between the inoculated and control plants indicate the function of the isolated plant DNA.

A still further aspect of the present invention is a method of screening plant DNA sequences for function. The method comprises preparing a silencing vector as described above, containing one or more DNA sequences identical to, or having substantial sequence similarity to, a plant gene (including fragments thereof). A test plant or a test plant tissue is then inoculated with the silencing vector and allowed to grow for a period of time, then compared to a non-inoculated or sham inoculated control plant or control plant tissue. Differences between the inoculated and control plant tissue indicate the function of the silenced plant gene.

In particular embodiments of the screening methods described herein, the same plant comprises the test plant and the control plant tissue.

These and other aspects of the invention are set forth in more detail in the description of the invention set forth below.

### BR1EF DESCRIPTION OF THE DRAWINGS

FIG. 1 shows the A and Be genetic components for TGMV and CbLCV. Panel A shows the TGMV A and B genetic components; each contains a common region that includes the origin of replication. AL1, AL2 and AL3 are viral genes needed for replication and gene expression. The AR1 gene encodes the coat protein, which can be replaced with the insertion of foreign DNA at the multiple cloning site (MCS). The B component encodes two movement proteins, BL1 and BR1. The TGMV B component contains a unique Xbal site, 15 bp downstream of the BR1 ORF stop codon, engineered for insertion of foreign sequences. CR indicates the common region. Panel B shows the CbLCV A and B genetic components; each contains a common region that includes the origin of replication. AL1, AL2 and AL3 are viral genes needed for replication and gene expression. The AR1 gene encodes the coat protein, which can be replaced with the insertion of foreign DNA at the multiple cloning site (MCS). The B component encodes two movement proteins, BL1 and BR1. The CbLCV B component contains a naturally-occurring, unique HincII site upstream of the BR1 stop codon used for insertion of foreign sequences. CR indicates the common region.

FIG. 2 shows the immunolocalization of PCNA in silenced meristems. Apical meristems from TGMV A::790su/B::122PCNA infected plants (Panels A-D), TGMV A::790su/B infected plants (Panels E, F, H, I) or A/B infected plants (Panel G) fixed 4 weeks post inoculation, vibratome-sectioned, and localized for PCNA protein (reddish-brown precipitate, Panels B, D, E, G, I) or DNA (DAPI, Panels A, C, F, H). Apical meristems from plants silenced for PCNA (Panels A-D) lack PCNA staining in large areas of the meristem. Arrows show PCNA positive nuclei that appear dark under UV fluorescence because of precipitated stain. Apical meristems from su-silenced or wild type TGMV infected plants (Panels E, F, G) show random PCNA staining throughout the meristem, consistent with S phase expression. Axillary bud meristems from A::790su/B infected plants still contain PCNA (Panels H, I) although they were not actively dividing at the time of fixation. Bar=200 μm (Panels A-G) or 50 μm (Panels H, I).

FIG. 3 shows in situ hybridization of CbLCV in N. benthamiana and Arabidopsis. Viral DNA probes were labeled with digoxigenin using PCR. Plants were infected by bombardment, sectioned with a vibratome, and hybridized with probe. Arrows show infected nuclei outside of vascular tissue. Panel A shows an N. benthamiana stem cross section. Panel B shows an N. benthamiana leaf cross section. Panel C shows an Arabidopsis leaf cross section, DAPI stained to show the location of nuclei. Arrow shows area with infected (black) and healthy (blue) nuclei. Panel D shows an Arabidopsis leaf cross section under bright field microscopy to show digoxigenin labeling.

FIG. 4 shows in situ hybridization of silenced and wild type virus-infected tissue probed for viral DNA accumulation, detected by a digoxigenin-labeled DNA probe from TGMV A. (Panel A) Wild type TGMV-infected tissue is green and shows contiguous cells with nuclear accumulation of viral DNA. (Panel B) TGMV A::790su/B infected leaf tissue lacks chlorophyll. Arrow shows viral DNA. (Panel C) Same as (Panel B), UV fluorescence shows plant nuclei stained with DAPI. The digoxigenin label caused precipitation of stain over the infected nucleus (arrow) reducing the DAPI signal. Other nuclei lack visible precipitate.

FIG. 5 shows a N. benthamiana plant inoculated with a TGMV B containing a 154-bp fragment of su in conjunction with either a wild type TGMV A component (plant on right) or with a mutant of TGMV A that confers a phloem-limited phenotype (plant on left). Plants were viewed under white light.

FIG. 6 depicts the N. benthamiana magnesium chelatase gene (su) cDNA, which includes 23-bp of upstream, non-coding sequence and a 1392-bp coding sequence. The 51-bp fragment was used to make vector TGMV A::51su. Vectors TGMV A::92su and TGMV B::154su contain the 92-bp fragment, corresponding to nt 781-873. The 154-bp, corresponding to nt 785-939, was used to make vector TGMV B::154su. Vector NBsul455 contains a 479-bp fragment, the corresponding to nt 936-1415. A 935-bp fragment, corresponding to nt 0-935 was used to make pNB935.

FIG. 7 shows photographs of transgenic N. benthamiana after inoculation with a Tomato golden mosaic virus (TGMV A) vector containing a 51-bp (Panel A) and 92-bp (Panel B) fragment of the su gene, which results in yellowing of green tissue when used for silencing. Both fragments were inserted into the A component, replacing the coat protein gene AR1, and subsequently co-introduced with wild type B component into N. benthamiana.

FIG. 8 shows photographs of transgenic N. benthamiana after inoculation with a Tomato golden mosaic virus (TGMV) B component containing either a 92-bp (Panel A), 154-bp (Panel B), 479-bp (Panel C), or 935-bp (Panel D) fragment of the su gene. All fragments were cloned into the same location of the B component, just downstream of the BR1 stop codon but upstream of the polyadenylation signal sequence. TGMV B vectors were co-introduced with wild type A component into N. benthamiana. Individual leaves are shown in Panels B-D to show a closer view of symptoms and silencing. Photographs were taken at approximately 28 days post-inoculation.

FIG. 9 shows that insertion of a large foreign DNA in the TGMV B vector is destabilizing. DNA was isolated from plants 4 weeks post inoculation with TGMV A/B::18OPCNA or A/B::180PCNAtr, containing a tandem direct repeat of a 180-bp PCNA fragment. Upper panel (A) shows that viral DNA accumulation in new growth of plants inoculated with a single 180-bp insert was low compared to plants inoculated with the tandem repeat (360-bp insert). Accumulation of viral DNA from plants inoculated with the B component vector and wild type A, EV (empty vector) was higher than the same vector with insert DNA. Lower panel (B) shows PCR products spanning the inserted fragment from each of the plants in the upper plant. The 180-bp insert was stable whereas the tandem repeat (360-bp insert) was deleted. Control lanes included+lane; PCR template was the B component plasmid DNA, TGMV B::18OPCNA, —lane; template consisted of wild type B plasmid DNA (vector without PCNA insert), P; PCR template DNA isolated from a healthy plant.

FIG. 10 shows silencing of Ch42 in Arabidopsis with CbLCV A::Ch42. Plants were grown in soil under short days to promote vegetative growth. Following bombardment with CbLCV AR1 deletion (empty vector) or CbLCV A::CH42, they were transferred to higher light, long days where they developed anthocyanin. Three weeks after bombardment, silencing appeared in CbLCV A::CH42 transformed plants. (yellow tissue; Panel C and D). There was no chlorosis in the empty vector control (Panel A and B). Wild type CbLCV does produce extensive chlorosis in leaves (Panel F), but the chlorosis is distinguishable from silencing (more brown-white than yellow-white). Panel E shows a mock inoculation.

FIG. 11 shows Arabidopsis after transformation of plants at the 4-leaf stage. Panel A shows an Arabidopsis plant transformed with CbLCV A::CH-42 and a wild type CbLCV B component. The arrow points to systemic silencing. Panel B shows an Arabidopsis plant transformed with CbLCV A::CH-42 alone; yellow spots are seen, but systemic silencing is absent due to the inability of the A component to move without the B component. Panels C and D show Arabidopsis plants transformed with wild type CbLCV A component and recombinant CbLCV B::CH-42. There is evidence of silencing in the transformed leaves, but not in the upper leaves yet. The BR1 gene in this construct was mutated inadvertently therefore possibly restricting the movement of the B component. All plants were photographed 12 days post infection.

FIG. 12 shows N. benthamiana inoculated with a TGMV A/B::122PCNA. Symptoms developed in lower leaves but primary growth and stem elongation ceased in upper parts of the plant. This plant never recovered primary growth. One flower is visible that may have been formed at or before movement of silencing into the apical area.

FIG. 13 shows silencing of the Ch42 locus in two different ecoyptes of Arabidopsis plants transformed with a 144-bp fragment of Ch42. The transformation event was conducted on plants at the 4-leaf stage of growth on plates thus not all plants were silenced. Panels A-D and E show Columbia ecotype and Panel F shows ecotype Landsberg. Panels A and D show the same plants from a different view.

FIG. 14 shows an example of silencing of su and gfp using the TGMV B vector in an N. benthamiana plant expressing GFP from an CaMV 35S promoter. GFP-transgenic plants were transformed, in conjunction with a wild type TGMV A component, with the TGMV B vector harboring a 140-bp fusion gene consisting of 58-bp of su and 82-bp of gfp (Left plant, Panels A and B). As a control, GFP-transgenic plants were infected with wild type TGMV A and B (Right plant, Panels A and B). Plants were photographed under UV illumination (Panel A) or white light (Panel B).

FIG. 15 shows silencing of two endogenous genes was achieved from DNA fragments carried in different TGMV component vectors. Variegation occurred in leaves that were partly expanded at the time of inoculation, however very little stem elongation was evident in new growth (Panel A). Plant is shown 3.5 weeks post-inoculation with TGMV A::790su/B::122PCNA. Plant (Panel B) inoculated with TGMV A::790su/B::122PCNA and pruned (arrow) two weeks after inoculation showed silencing in axillary buds. PCNA silencing is evidenced by reduced stem elongation and aberrant leaf formation. The two axillary outgrowths show different degrees of su silencing with one cluster of leaves (right) showing almost no chlorophyll. Note circular yellow spots in inoculated lower leaves (black arrow).

FIG. 16 shows silencing of Ch42 and gfp using the CbLCV vector in an Arabidopsis plant expressing GFP from a CaMV 35S promoter. Panel A shows a healthy 35S-gfp plant containing no virus. Panel B shows an Arabidopsis plant transformed with a CbLCV A (-AR1) mutant as an experimental control. The empty vector control caused an increase in GFP expression compared to the healthy plant. This has been noted for TGMV infections of transgenic N. benthamiana. Panel C shows an Arabidopsis plant transformed with CbLCV A::GFP, containing a 400-bp GFP fragment in the A component. Panel D shows an Arabidopsis plant inoculated with CbLCV::CH42; CbLCV A component with a 364-bp Ch-42 insert. Panel E shows an Arabidopsis plant inoculated with CbLCV::CH42-GFP, a fusion of the 400-bp GFP fragment and 364-bp Ch-42 fragment cloned into CbLCV A. All plants were viewed in the presence of white light to evaluate the absence of chlorophyll (yellow tissue) and UV light to evaluate the presence of GFP protein (yellow fluorescence).

FIG. 17 shows an agarose gel demonstrating replication of the viral vector in systemically-infected leaves. The blot was probed with CbCLV DNA. Lanes 1-10 is DNA isolated from Canola leaves and subsequently digested with DpnI. Lanes 11-13 show high molecular weight undigested DNA from lanes 2, 4, and 7.

### DETAILED DESCRIPTION OF THE INVENTION

Except as otherwise indicated, standard methods may be used for the production of cloned genes, expression cassettes, silencing cassettes, vectors, proteins and protein fragments, and transformed cells and plants according to the present invention. Such techniques are known to those skilled in the art (see e.g., SAMBROOK et al., EDS., MOLECULAR CLONING: A LABORATORY MANUAL 2d ed. (Cold Spring Harbor, NY 1989); F. M. AUSUBEL et al, EDS., CURRENT PROTOCOLS IN MOLECULAR BIOLOGY (Green Publishing Associates, Inc. and John Wiley & Sons, Inc., New York); J. DRAPER et al., EDS., PLANT GENETIC TRANSFORMATION AND GENE EXPRESSION: A LABORATORY MANUAL, (Blackwell Scientific Publications, 1988); and S. B. GELVIN & R. A. SCHILPEROORT, EDS., INTRODUCTION, EXPRESSION, AND ANALYSIS OF GENE PRODUCTION IN PLANTS.

The terminology used in the description of the invention herein is for the purpose of describing particular embodiments only and is not intended to be limiting of the invention. As used in the description of the invention and the appended claims, the singular forms “a” “an” and “the” are intended to include the plural forms as well, unless the context clearly indicates otherwise. All publications, patent applications, patents, and other references mentioned herein are incorporated by reference in their entirety.

The present investigations demonstrate that DNA carried on episomes can silence active, chromosomal gene expression, and that DNA plant viruses (and particularly geminiviruses) can provide a mechanism for the suppression of gene expression in intact plants (preferably, systemic suppression). The present inventors show that a nuclear-localized DNA virus (e.g., a geminivirus) carrying sequences complementary to (i.e., homologous to, or having substantial sequence similarity to) chromosomal genes can effect silencing of the chromosomal gene.

The present inventors determined that silencing of plant genes can be triggered by homologous sequences carried by a DNA episome, such as a geminivirus construct. Where the episome is capable of spreading from cell to cell in a plant (or capable of producing a diffusible silencing factor), systemic silencing of chromosomal genes can be achieved. Moreover, in at least some instances, silencing may be achieved in the absence of detectable transcription of the homologous gene sequence.

Previous reports have demonstrated gene expression from geminivirus-derived episomes (reviewed in Timmermans et al., Annu. Rev. Plant Physiol. 45, 79, (1994)). A direct correlation between episome copy number and gene expression was shown in cultured cells for TGMV carrying the neo gene (Kanevski et al., Plant J. 2, 457, (1992)). The present experiments differ in that there was no selection for gene expression, there was homology (i.e., sequence identity or substantial sequence similarity) between the episomal and chromosomal sequences, and only partial copies of the silenced endogenous genes (su, pcnA or gfp genes) were carried in episomes. Moreover, the sequences inserted into the inventive silencing vector may be in the sense or anti-sense orientation. Unlike the expression vector described by U.S. Pat. No. 6,077,992, the silencing vectors of the invention do not typically stably integrate into the plant genetic material and are not expressed in the seed. The present invention demonstrates silencing of chromosomal gene expression by episomal DNA; more specifically, the ability to silence endogenous gene expression systemically in a plant using a plant virus construct is demonstrated.

There have also been reports of gene suppression in plants using Agrobacteria carrying sense and/or anti-sense sequences homologous to plant genes (Seymour et al., Plant Molecular Biology 23, 1 (1993); Jones et al, (1996) Down-regulation of two non-homologous endogenous genes with a single chimeric gene construct, In: Grierson D., Lycett G W, Tucker G A (eds) Mechanisms and applications of gene silencing, Nottingham University Press, UK, pp. 85; Jones et al., Planta 204, 499 (1998)). The approaches employed in these investigations have several disadvantages as compared with the present invention. For example, these methods typically require stable transformation followed by regeneration of entire plants. In contrast, the present invention permits the silencing of gene expression in mature plants, without the need for stable transformation (i.e., the silencing vector remains episomal) of individual plant cells and subsequent regeneration of whole plants. Thus, the present invention may be more suitable for rapid screening of gene function, e.g., functional genomic approaches. Further, the present invention may be advantageously employed to assess gene function, particularly in the case where the target or targets of gene suppression confer a lethal phenotype (e.g., knockouts for the magnesium chelatase gene).

In addition, the vectors of the present invention replicate as non-integrating episomes, thereby avoiding position effects and reducing the likelihood of chromosomal rearrangements or other alterations to the plant chromosomes, both of which raise concerns in methodologies utilizing integrating vectors.

Moreover, the present methods also allow the suppression of plant gene expression without the modification of the germplasm.

Kjemtrup et al., (1998) Plant J. 14:91 and international patent publication WO 99/50429 describe gene silencing using geminivirus vectors. The investigations described herein demonstrate that insertion of homologous sequences into the B component of the bipartite geminivirus results in silencing (preferably, systemic silencing) of endogenous gene expression. Indeed, in some systems, geminivirus B component vectors give higher and/or more wide-spread silencing than do A component vectors. The present investigations have further discovered that surprisingly short sequences may be used to achieve silencing in plants. Moreover, the present invention provides the unexpected discovery that gene silencing, including systemic gene silencing, may be achieved by phloem-limited geminivirus (e.g., in cells outside of the phloem).

Geminiviruses and other DNA Viruses

The geminiviruses are single-stranded plant DNA viruses. They possess a circular, single-stranded (ss) genomic DNA encapsidated in twinned “geminate” icosahedral particles. The encapsidated ss DNAs are replicated through circular double stranded DNA intermediates in the nucleus of the host cell, presumably by a rolling circle mechanism. Viral DNA replication, which results in the simulation of both single and double stranded viral DNAs in large amounts, involves the expression of only a small number of viral proteins that are necessary either for the replication process itself or facilitates replication or viral transcription. The geminiviruses therefore appear to rely primarily on the machinery of the host for viral replication and gene expression.

Geminiviruses are subdivided on the basis of host range in either monocots or dicots and whether the insect vector is a leaf hopper or a white fly species. Monocot-infecting geminiviruses are typically transmitted by leaf hoppers and their genome comprises a single ss DNA component about 2.7 kb in size (monopartite geminivirus). This type of genome, the smallest known infectious DNA, is typified by wheat dwarf virus which is one of a number from the subgroup that have been cloned and sequenced. Most geminiviruses that infect dicot hosts are transmitted by the white fly and possess a bipartite genome comprising similarly sized DNAs (usually termed A and B) as illustrated by African cassava mosaic virus (ACMV), tomato golden mosaic virus (TGMV) and potato yellow mosaic virus. For successful infection of plants, both genomic components are required. Beet curly top virus occupies a unique intermediary position between the above two subgroups as it infects dicots but contains only a single genomic component equivalent to DNA A, possibly as a result of adaptation to leaf hopper transmission.

The bipartite subgroup contains only the viruses that infect dicots. Exemplary is the African Cassava Mosaic Virus (ACMV) and the Tomato Golden Mosaic Virus (TGMV). TGMV, like ACMV, is composed of two circular DNA molecules of the same size, both of which are required for infectivity. Sequence analysis of the two genome components reveals six open reading frames (ORFs); four of the ORFs are encoded by DNA A and two by DNA B. On both components, the ORFs diverge from a conserved 230 nucleotide intergenic region (common region) and are transcribed bidirectionally from double stranded replicative form DNA. The ORFs are named according to genome component and orientation relative to the common region (i.e., left versus right). The AL2 gene product transactivates expression of the TGMV coat protein gene, which is also sometimes known as “AR1”. Functions have not yet been attributed to some of the ORFs in the geminivirus genomes. However, it is known that certain proteins are involved in the replication of viral DNA (REP genes). See, e.g., Elmer et al., Nucleic Acids Res. 16, 7043 (1988); Hatta and Francki, Virology 92, 428 (1979).

The A genome component contains all viral information necessary for the replication and encapsidation of viral DNA, while the B component encodes functions required for movement of the virus through the infected plant. The DNA A component of these viruses is capable of autonomous replication in plant cells in the absence of DNA B when inserted as a greater than full-length copy into the genome of plant cells, or when a copy is electroporated into plant cells. In monopartite geminivirus genomes, the single genomic component contains all viral information necessary for replication, encapsidation, and movement of the virus.

The geminivirus A component carries the AL1 (also known as C1 or REP), the AL2 (also known as C2 or TRAP), AL3 (also known as C3 or REN), and AR1 (also known as V1 or coat protein) sequences. The geminivirus B component carries the BR1(also known as BV1) and BR1 (also known as BC1) sequences.

Little is known about the interaction of geminiviruses with their hosts. Because they replicate to high copy numbers in plant nuclei, they may have evolved mechanisms to evade homology sensing and silencing mechanisms. The present inventors have determined that insertion of plant DNA into the geminivirus genome can trigger gene silencing in the host plant.

As used herein, geminiviruses encompass viruses of the Genus Mastrevirus, Genus Curtovirus, and Genus Begomovirus. Exemplary geminiviruses include, but are not limited to, Abutilon Mosaic Virus, Ageratum Yellow Vein Virus, Bhendi Yellow Vein Mosaic virus, Cassava African Mosaic Virus, Chino del Tomato Virus, Cotton Leaf Crumple Virus, Croton Yellow Vein Mosaic Virus, Dolichos Yellow Mosaic Virus, Horsegram Yellow Mosaic Virus, Jatropha Mosaic virus, Lima Bean Golden Mosaic Virus, Melon Leaf Curl Virus, Mung Bean Yellow Mosaic Virus, Okra Leaf Curl Virus, Pepper Hausteco Virus, Potato Yellow Mosaic Virus, Rhynchosia Mosaic Virus, Squash Leaf Curl Virus, Tobacco Leaf Curl Virus, Tomato Australian Leaf Curl Virus, Tomato Indian Leaf Curl Virus, Tomato Leaf Crumple Virus, Tomato Yellow Leaf Curl Virus, Tomato Yellow Mosaic Virus, Watermelon Chlorotic Stunt Virus, Watermelon Curly Mottle Virus, Bean Distortion Dwarf Virus, Cowpea Golden Mosaic Virus, Lupin Leaf Curl Virus, Solanum Apical Leaf Curling Virus, Soybean Crinkle Leaf Virus, Chloris Striate Mosaic Virus, Digitaria Striate Mosaic Virus, Digitaria Streak Virus, Miscanthus Streak Virus, Panicum Streak Virus, Pasalum Striate Mosaic Virus, Sugarcane Streak Virus, Tobacco Yellow Dwarf Virus, Cassava Indian Mosaic Virus, Serrano Golden Mosaic Virus, Tomato Golden Mosaic Virus, Cabbage Leaf Curl Virus, Bean Golden Mosaic Virus, Pepper Texas Virus, Tomato Mottle Virus, Euphorbia Mosaic Virus, African Cassava Mosaic Virus, Bean Calico Mosaic Virus, Wheat Dwarf Virus, Cotton Leaf Curl Virus, Maize Streak Virus, and any other virus designated as a Gerninivirus by the International Committee on Taxonomy of Viruses (ICTV).

Badnaviruses are a genus of plant viruses having double-stranded DNA genomes. Specific badnavirus include cacao swollen shoot virus and rice tungro bacilliform virus (RTBV). Most badnavirus have a narrow host range and are transmitted by insect vectors. In the badnaviruses, a single open reading frame (ORF) may encode the movement protein, coat protein, protease and reverse transcriptase; proteolytic processing produces the final products.

Exemplary Badnaviruses include, but are not limited to Commelina Yellow Mottle Virus, Banana Streak Virus, Cacao Swollen Shoot Virus, Canna Yellow Mottle Virus, Dioscorea Bacilliform Virus, Kalanchoe Top-Spotting Virus, Piper Yellow Mottle Virus, Rice Tungro Bacilliform Virus, Schefflera Ringspot Virus, Sugarcane Bacilliform Virus, Aucuba Bacilliform Virus, Mimosa Baciliform Virus, Taro Bacilliform Virus, Yucca Bacilliform Virus, Rubus Yellow Net Virus, Sweet Potato Leaf Curl Virus, Yam Internal Brown Spot Virus, and any other virus designated as a Badnavirus by the International Committee on Taxonomy of Viruses (ICTV).

Caulimoviruses have double-stranded circular DNA genomes that replicate through a reverse transcriptase-mediated process, although the virus DNA is not integrated into the host genome. As used herein, Caulimoviruses include but are not limited to Cauliflower Mosaic Virus, Blueberry Red Ringspot Virus, Carnation Etched Ring Virus, Dahlia Mosaic Virus, Figwort Mosaic Virus, Horseradish Latent Virus, Mirabilis Mosaic Virus, Peanut Chlorotic Streak Virus, Soybean Chlorotic Mottle Virus, Strawberry Vein Banding Virus, Thistle Mottle Virus, Aquilegia Necrotic Mosaic Virus, Cestrum Virus, Petunia Vein Clearing Virus, Plantago Virus, Sonchus Mottle Virus, and any other virus designated as a Caulimovirus by the International Committee on Taxonomy of Viruses (ICTV).

The Nanoviruses have single-stranded circular DNA genomes. As used herein, Nanoviruses include but are not limited to Banana Bunchy Top Nanavirus, Coconut Foliar Decay Nanavirus, Faba Bean Necrotic Yellows Nanavirus, Milk Vetch Dwarf Nanavirus, and any other virus designated as a Nanovirus by the International Committee on Taxonomy of Viruses (ICTV).

Episomally-Mediated Gene Silencing.

The present invention provides methods of silencing endogenous plant genes (as defined below) using DNA episomes, and provides constructs for use in such methods. The episomal DNA carries one or more heterologous DNA sequences, where each sequence is homologous (i.e., has substantial sequence homology) to an endogenous plant gene(s) to be silenced, or homologous to a fragment of the endogenous plant gene to be silenced. The DNA episomes are preferentially able to replicate to multiple copy numbers in plant nuclei; where systemic silencing is desired, the episome is preferably able to move from cell-to-cell in the plant or to induce the movement of a diffusible suppression factor (or “silencing factor”), in order to enter and affect cells remote from the initial point of inoculation. The gene silencing may result in an altered phenotype; “altered phenotype” as used herein includes alterations in characteristics that can be visually observed (e.g., color), measured (e.g., average height or other growth characteristics) or biochemically assessed (e.g., presence of amounts of target gene products, including RNA, protein, or peptide products, or downstream biochemical pathway products). Visual observations include observations that employ microscopic and spectroscopic techniques.

As used herein, an “endogenous” plant gene refers to a plant gene found in the chromosomal DNA of the plant, i.e., a gene that occurs naturally in the plant nuclear or plastid genomes, preferably, the nuclear genome. In particular embodiments, the invention may be used to silence a transgene that has been integrated into the plant genetic material, e.g, by Agrobacterium-mediated transformation or ballistic bombardment). For example, a gene encoding a reporter protein or peptide may be introduced into the plant and serve as a marker in gene suppression studies.

As used herein, the term “silenced” or “gene silencing” refers to a reduction in the expression product of a target gene. Silencing may occur at the transcriptional or post-transcriptional level. Silencing may be assessed on the cellular level (i.e., by assessing the gene products in a particular cell), or at the plant tissue level (assessing silencing in a particular type of plant tissue) or at the level of the entire plant. Silencing may be complete, in that no final gene product is produced, or partial, in that a substantial reduction in the accumulation of gene product occurs. Such reduction may result in accumulations of gene product that are less than 90%, less than 75%, less than 50%, less than 30%, less than 20%, less than 10%, less than 5%, or even less than that produced by non-silenced genes.

As used herein, “systemic silencing” refers to the silencing of genes in plants, plant cells, or plant tissues, where gene silencing occurs in cells that are remote from the site of initial inoculation of the DNA-silencing episome. Applicants do not wish to be held to a single theory of systemic silencing; systemic silencing may occur by the replication and cell-to-cell movement of DNA constructs, or by the movement of a mobile silencing factor. Systemic silencing does not require that every tissue or every cell of the plant be affected, as the effects and extent of silencing may vary from tissue to tissue, or among cells.

It is not necessary that the episomal silencing constructs of the invention include viral movement protein genes to accomplish gene silencing. The present inventors have determined that episomal-mediated gene silencing may be achieved in the absence of the viral movement proteins.

As a further aspect, the present invention provides the novel discovery that gene silencing, and preferably systemic gene silencing, may be achieved with phloem-limited geminivirus silencing vectors in cells and tissues outside of the phloem. This finding is of interest, as most characterized geminiviruses are believed to be phloem-limited. Indeed, in some systems, higher levels of silencing may be advantageously achieved with phloem-limited geminivirus vectors. While not wishing to be held to any particular theory of the invention, it appears that a viral anti-silencing signal may be obviated by limiting the vector to the phloem, thereby resulting in higher levels of gene suppression. Alternatively, it appears that restricting the virus to the phloem tissue may advantageously reduce the pathology of the virus in the host plant.

Accordingly, in particular preferred embodiments of the invention, the silencing vector is phloem-limited, as that term is understood in the art. The silencing vector may be a geminivirus silencing vector that is derived from a geminivirus genomic component that is naturally phloem-limited (e.g., derived from the A component or B component of a phloem-limited geminivirus). Alternatively, the silencing vector may be phloem-limited as a result of a phloem-limiting mutation, e.g., the Ala5 mutation in the TGMV genomic A component (in the AL1 gene) as described in co-pending U.S. application Ser. No. 09/289,346 to Hanley-Bowdoin et al. This mutation results in a KEE→AAA mutation at amino acids 143-146 in the AL1 protein (within helix 4 of the oligomerization domain).

In still other preferred embodiments, the silencing vector comprises a Leu→ALA mutation at amino acid residue 148 in the TGMV genomic A component (in the AL1 gene). This mutation results in phloem limitation of the virus, and also appears to result in higher levels of DNA replication.

Those skilled in the art will appreciate that these mutations (i.e., Ala5 and Leu₁₄₈→Ala₁₄₈ may be incorporated into the corresponding positions of the genomic DNA of other geminiviruses.

As still a further aspect, the present invention provides gene silencing vectors in which a heterologous DNA sequence having substantial sequence similarity to an endogenous plant gene (including gene fragments) is inserted in the 3′ non-coding region of a viral gene, so that the DNA sequence is co-transcribed with the viral gene, but is not translated. In some systems, increased spread of silencing is achieved using this method. Gene silencing vectors from any plant DNA virus may be modified to carry heterologous DNA sequences according to this method (preferably, geminivirus silencing vectors). For example, the heterologous DNA sequence can be inserted downstream of genes in the A component (AL1, AL2, AL3, AR1) or the B component (BLI, BR1) of a bipartite geminivirus, the single component of monopartite geminiviruses, or any of the genes of a plant DNA virus such as a nanovirus, badnavirus, or caulimovirus.

As used herein, the term “DNA silencing episome” or “DNA silencing vector” refers to a DNA construct capable of replicating within a host cell, and carrying one or more heterologous (or “recombinant”) DNA sequences, where each sequence is substantially similar or identical in nucleotide sequence to an endogenous host plant gene (including fragments of the plant gene). Typically, and preferably, the DNA silencing episomes of the invention are localized to the nucleus of the host cell.

As used herein, a heterologous sequence that has “substantial sequence similarity to an endogenous plant gene” has substantial sequence similarity at the nucleotide level to an endogenous plant gene, as described above, or a fragment of the plant gene, including the coding sequences of the gene and non-coding sequences (including intron sequences and 5′ and 3′ untranslated sequences). A “fragment” of a plant gene is a polynucleotide sequence that is shorter in length than the full-length gene, and may be a sequence of at least 10, 20, 30, 50, 75, 100, 150, 200, 500, 700, or more, contiguous nucleotides.

By “substantial sequence similarity” it is meant that the heterologous DNA sequence is of sufficient sequence similarity to the endogenous gene that silencing of the endogenous gene occurs upon introduction of the episome. Such DNA sequences are substantially similar in nucleotide sequence to the endogenous sequence (including fragments thereof) to be silenced; the heterologous DNA sequence may have from 60% sequence similarity, 70% sequence similarity, 75% sequence similarity, 80% sequence similarity, 85% sequence similarity, 90% sequence similarity, 95% sequence similarity, or even 97% or 98% sequence similarity, or more, to the target endogenous sequence (or a fragment thereof).

As is known in the art, a number of different programs can be used to identify whether a nucleic acid has sequence identity or similarity to a known sequence. Sequence identity and/or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402.

A “% sequence similarity” as used herein indicates the percentage of nucleotide residues in the heterologous sequence that are identical with the nucleotide residues in the target endogenous plant gene sequence (including fragments thereof).

As used herein, the term “heterologous DNA” contained on the DNA silencing episome refers to DNA that is not naturally found in conjunction with the DNA episomal construct, i.e., that has been introduced by genetic engineering techniques. The heterologous DNA is of a size sufficient to silence the endogenous target gene (see below). The heterologous DNA may be in sense or antisense orientation, and may be frame-shifted as compared with the coding sequence. One skilled in the art will be able, using techniques available in the art and without undue experimentation, to test and select gene fragments for their ability to induce silencing when used in the present methods.

The DNA silencing episome described above may comprise multiple heterologous sequences (e.g., two, three, four, five, six or even more heterologous DNA sequences as described above) that are identical to or substantially similar to two or more endogenous plant genes (including gene fragments). According to this embodiment, the present invention may be used to silence two or more endogenous plant genes (e.g., two, three, four, five, six or even more endogenous plant genes). In preferred embodiments, two or more non-homologous endogenous plant genes are silenced (i.e., the genes are not part of a gene family). In other words, the present invention may be employed to suppress two unrelated genes.

Those skilled in the art will appreciate that in this embodiment, one of the nucleotide sequences may also effect silencing of more than one endogenous plant gene within a gene family. Likewise a single heterologous DNA sequence may silence more than one endogenous plant gene, typically homologous plant genes.

The two or more nucleotide sequences may be in the sense or antisense orientation, or a mixture thereof (i.e., some sequences in the sense and some sequences in the antisense orientation), and may further be frame-shifted as compared with the coding sequence. Each of the homologous sequences may be operably associated with a different promoter. Alternatively, two or more of the sequences, or even all of the sequences, will be operably associated with a single promoter (e.g., a geminivirus AR1, BR1 or BL1 promoter). As described hereinbelow, in particular embodiments, one or more of the nucleotide sequences is not operably associated with a promoter. Typically, however, an increased spread of gene silencing will be observed if the heterologous DNA sequence is operably associated with a promoter that drives transcription of the sequence (in either the sense or antisense direction).

Multiple gene silencing may be advantageously employed to alter the phenotype of a plant, as described in more detail hereinbelow. Silencing of multiple genes may be necessary to alter a single phenotypic trait; alternatively, multiple genes may be suppressed to modify more than one phenotypic trait in the plant.

A preferred recombinant episomal silencing construct contains one or more heterologous DNA sequences, which may be any sequence having sequence identity to, or substantial sequence similarity to, an open reading frame of an endogenous gene encoding a polypeptide of interest (for example, an enzyme). Alternatively or in addition, the heterologous nucleotide sequence may be identical to or have substantial sequence similarity to an endogenous, genomic sequence, where the genomic sequence may be an open reading frame, an intron, a noncoding 5′ or 3′ sequence, or any other sequence which inhibits transcription, messenger RNA processing (for example, splicing), or translation.

In one particular embodiment, the inventors have determined that DNA constructs, such as the geminivirus constructs described herein, are capable of silencing endogenous plant promoters, where the DNA construct introduced into the target plant carries DNA having sequence identity to (or substantial sequence similarity to) an endogenous promoter sequence. Thus, where a family of homologous genes exists, but the associated promoters differ, selective silencing of one member of the gene family may be achieved by suppressing its promoter, using episomal constructs of the present invention. Such promoters may be tissue-specific (e.g., promoters associated with leaf-specific actins, as compared to actins expressed in other plant tissues) or developmentally regulated promoters. Examples of such promoters are known in the art.

In other particular embodiments, the heterologous DNA sequence (s) has substantial sequence similarity to a gene (including a fragment thereof), encoding a non-translated RNA molecule. Exemplary non-translated RNA molecules include but are not limited to ribozymes, transfer RNA, ribosomal RNA, and snRNA molecules.

The heterologous DNA segment carried by the silencing construct may represent only a fragment of the endogenous gene to be silenced or, alternatively, the entire gene (which may only include coding regions or may further include non-coding regions, such as introns and 5′ and 3′ untranslated sequences). The present inventors have surprisingly shown that relatively small fragments of genes, in either the sense or antisense orientation, are sufficient to induce silencing.

There are no particular lower or upper limits to the length of the heterologous DNA sequences carried by the silencing vector. The fragment may be significantly shorter than the entire gene. The present inventors have made the surprising discovery that relatively short sequences may be used to effect gene silencing. The nucleotide sequence(s) may be as short as about 150, 100, 75, 50, 40, 30, or even 20 nucleotides in length, or even shorter, as long as the nucleotide sequence(s) provides for a desired level of gene silencing. There is no particular upper limit to the length of the nucleotide sequence or combined length of multiple sequences, subject to the carrying capacity of the silencing vector. e.g., the heterologous DNA sequence(s) may be as long as 150, 250, 500, 800, 1000 or even 1500 nucleotides in length.

To illustrate, it is typical for bipartite geminiviruses that episomal movement will be restricted as the size increases significantly beyond 2.9 kb. Accordingly, for vectors constructed from bipartite geminiviruses, it is preferred that the size of the episome is less than about 3.5 kb, more preferably, less than about 3.2 kb, still more preferably, less than about 3.0 kb. Alternatively stated, it is preferred that the silencing vector is approximately the size of a wild-type geminivirus genomic component, e.g., approximately 2.5-2.6 kb for a bipartite genomic component.

In general, it is preferred that the silencing vector is approximately 80% to 120%, more preferably approximately 90% to 110%, still more preferably approximately 95% to 105% the size of the wild type monopartite or bipartite genomic component.

As still a further alternative, in the case of “pop out” vectors as described below, i.e., a larger construct (typically, a shuttle vector) from which the geminivirus silencing vector excises itself, it is preferred that the “pop out” geminivirus vector have a total size as described in the previous paragraph. The larger construct (e.g., a shuttle vector) that is initially inoculated into the plant, will typically be substantially larger than the wild-type geminivirus genomic component.

Further, the size restrictions on the heterologous nucleotide sequence(s) may depend on the site of insertion or replacement within the geminivirus genome. For example, typically about 800 nucleotides of the geminivirus coat protein may be replaced by heterologous DNA. In contrast, heterologous DNA sequences inserted or replaced within the B component, in particular the 3′ non-coding sequences following the stop codon of the BR1 or BL1 gene, are preferably less than about 300 nucleotides in length, more preferably less than about 250 nucleotides in length, still more preferably less than about 200 nucleotides in length, and yet more preferably less than about 150 nucleotides in length. Those skilled in the art will appreciate that certain sequences may be deleted from the B component (e.g., in the intergenic region) to increase the capacity for foreign sequences.

The heterologous DNA sequences of this invention may be synthetic, naturally-derived, or combinations thereof. Methods of producing recombinant DNA constructs are well known in the art.

In particular embodiments, the DNA silencing episomes of the present invention need not have a promoter operably linked to the heterologous DNA segment therein. Use of a silencing construct carrying a heterologous DNA segment as described above, where that DNA segment is not operably linked to a promoter in the DNA construct, may still result in silencing of an endogenous plant gene(s), and may further result in systemic silencing. Use of a promoter operably linked to the heterologous DNA in the silencing construct, however, is preferred and may increase the extent of the systemic silencing. Any promoter known in the art may be used, with plant promoters being preferred. Typically, however, the heterologous DNA segment is operatively associated with a native viral promoter.

The heterologous DNA sequence may further be associated with other transcriptional control sequences, as are known, in the art (e.g., enhancer sequences, transcriptional termination sequences, and the like). According to the present invention, it is not necessary that the heterologous DNA sequences be transcribed or translated, however, they may be (e.g., if inserted into a viral gene). Moreover, it appears that the spread of gene silencing is increased if the heterologous DNA sequence is transcribed by the plant host cell.

In preferred embodiments, the present invention utilizes DNA episomes based on plant viral genomes. A particularly preferred embodiment utilizes episomes based on geminivirus genomes. Additional plant DNA viruses include the Caulimoviruses, the Badnaviruses, and the Nanoviruses, as described above.

Novel recombinant geminivirus constructs including silencing vectors, expression vectors (e.g., to express an antisense sequence or relatively small peptide from the B genomic component), and transfer vectors (e.g., shuttle vectors) are provided. The present geminivirus constructs, when transfected into a plant cell, act to silence a gene already present in the plant cell. The gene to be silenced may be an endogenous plant gene, or a gene or DNA sequence that has previously been artificially introduced into the plant cell. The present geminivirus constructs further provide a method for the systemic silencing of a gene in a plant, for example, by providing both the A and B genome components of the geminivirus to the subject plant.

The present invention also provides “binary” silencing vectors that comprise regions from both the A and B genomic components of a bipartite geminivirus.

Where systemic silencing is desired, the construct is preferably capable of both replication in the host cell, and cell-to-cell movement (either of the DNA construct or a silencing factor). In the case of a bipartite geminivirus, this may be accomplished by using a binary vector, by co-introducing the A and B components, or by stably transforming the host plant to express the replication or movement proteins.

According to preferred embodiments of the present invention, the silencing vector comprises a geminivirus genomic component comprising one or more heterologous DNA sequences (as described above), where each of the heterologous DNA sequences has substantial sequence similarity to an endogenous plant gene(s).

In one particular preferred embodiment, the geminivirus genomic component is a geminivirus A genomic component. Heterologous DNA may replace any coding or non-coding region that is nonessential for the present purposes of gene silencing, or may be inserted just downstream of an endogenous viral gene, e.g., such that the viral gene and heterologous DNA are cotranscribed. In particular preferred embodiments, one or more heterologous nucleotide sequences may be inserted into or replace (preferably, replace) a segment of the sequence encoding the geminivirus coat protein (i.e., AR1 gene) or the common region. With respect to the common region, it is preferred that the heterologous DNA sequences are not inserted into or replace the Ori sequences or the flanking sequences that are required for viral DNA replication.

In other particular preferred embodiments, the vector further comprises geminivirus genes encoding the movement proteins (e.g, BR1 and/or BL1 genes). In alternative embodiments, both the geminivirus A and B components are carried by a single construct. The heterologous DNA sequence(s) may be inserted into or replace sequences within the B component as described below. For example, one or more heterologous DNA sequences may be inserted into the coding or 3′ non-coding regions of the BR1 and/or BL1 genes, the B component intergenic region, or the common region, as described further hereinbelow.

It will be appreciated by those skilled in the art, that the geminivirus constructs of the invention may be “hybrids” or “pseudorecombinants”, i.e., include sequences from two or more different geminiviruses or genomic components from different geminiviruses, respectively (see, e.g., Hill et al., (1998) Virology 250:283; Sung et al. (1995) J. Gen. Virol. 76:2809). Likewise, in the methods of the present invention, plants may be inoculated with genomic components (i e., A and B) from different geminiviruses, or with constructs carrying genes from different geminiviruses, as long as suitable levels of silencing according to the invention are achieved. In general, geminivirusesin which the A and B genomic components are from the same geminivirus are preferred.

In other preferred embodiments, the silencing vector comprises a geminivirus B component. Heterologous DNA may replace any coding or non-coding region that is nonessential for the present purposes of gene silencing, or may be inserted downstream of an endogenous viral gene such that the viral gene and heterologous DNA are cotranscribed. In particular embodiments, the heterologous DNA sequences may be inserted into or replace a segment of the 3′ non-coding sequences following the stop codon of the BR1 and/or BL1 genes, i.e., the sequence is 3′ of the stop codon and 5′ of the poly-A sequence so that the sequence is co-transcribed with the BR1 or BL1 gene, but is not translated. Alternatively, the heterologous DNA sequence(s) are inserted into or replace a portion of the coding region of the BR1 and/or BL1 genes, although systemic silencing may be reduced. As a further alternative, the heterologous DNA sequence(s) may be inserted into or replace a segment of the intergenic region between the BR1 and BL1 genes. As described above with respect to the A component, the heterologous DNA sequence(s) may be inserted into the common region of the B component.

In particular embodiments, the silencing construct further comprises the geminivirus genes encoding the replication proteins, e.g., the AL1, AL3 and/or AL2 genes. Constructs encoding the AL1 and/or AL3 genes are preferred. As described above, the silencing vector may be a binary construct comprising both a geminivirus A component and geminivirus B component. The heterologous DNA sequence(s) may be inserted into or replace sequences within the A component as described above.

An alternative preferred DNA silencing construct comprises an origin of replication from a plant DNA virus, preferably from a plant DNA virus such as a geminivirus. The construct further preferably includes DNA encoding any proteins necessary for replication of the DNA construct in a plant cell. In one particular embodiment, the silencing vector comprises geminivirus AL1, AL2, and AL3 genes, preferably, the AL1 or AL3 genes, more preferably, both the AL1 and AL3 genes. Additionally, or alternatively, the silencing vector may comprise the geminivirus AR1 gene. The origin of replication and DNA encoding necessary replication proteins may be obtained from the same geminivirus species; alternatively, the origin of replication may be from one geminivirus species and the replication proteins from a different geminivirus species. The construct further includes one or more heterologous DNA segments identical to, or having substantial sequence similarity to, an endogenous plant gene(s) to be silenced (or fragments thereof, as described above).

In another preferred embodiment, the silencing vector further comprises a geminivirus BR1 and/or BL1 gene, preferably both. The construct may further include the intergenic region from the B component.

One or more heterologous nucleotide sequences may be inserted into or replace segments of the coding and non-coding regions of the geminivirus A and B genomic components, as described above. Alternatively, a heterologous DNA sequence(s) may be inserted into the vector outside of the viral sequences.

An alternative preferred DNA silencing construct comprises an origin of replication from a plant DNA virus, preferably from a plant DNA virus such as a geminivirus. The construct further preferably includes DNA sequences encoding proteins required for viral movement, preferably, geminivirus sequences, more preferably, the geminivirus BR1 and/or BL1 genes. The origin of replication and DNA encoding the movement proteins may be obtained from the same geminivirus species; alternatively, the origin of replication may be from one geminivirus species and the replication proteins from a different geminivirus species. The construct further includes one or more heterologous DNA segments identical to, or having substantial sequence similarity to, an endogenous plant gene(s) to be silenced (or fragments thereof, as described above).

According to this embodiment, it is further preferred that the construct encode sequences required for replication of the construct in a plant cell. Preferably, the replication sequences are from a geminivirus, e.g., the geminivirus AL1, AL2, and AL3 sequences.

Those skilled in the art will appreciate that the total size of the silencing vector is approximately the size of a wild-type geminivirus genomic component, as described above.

The present invention also provides shuttle vectors which acts as a transfer vehicle for the silencing vector. The shuttle vector will typically replicate in a non-plant cell, e.g., a bacterial, yeast, or animal (e.g., insect, avian or mammalian) cell. Preferably, the shuttle vector replicates in bacterial cells. More preferably, the shuttle vector is a plasmid that replicates in bacterial cells (e.g, derived from pUC or an Agrobacterium Ri or Ti plasmid). In one particular embodiment of the invention, the geminivirus silencing vector is delivered in a shuttle plasmid, from which the geminivirus sequences excise themselves upon introduction into the plant cell. The shuttle vector may be introduced into the plant cell by any method known in the art, e.g., inoculation with Agrobacterium (as described below).

In some instances, introduction of the geminivirus silencing construct into the plant may cause pathology (e.g., disease, loss of viability, and the like) in the plant. Accordingly, it is preferred that the silencing vectors described herein comprise geminivirus genomic components (or alternatively, geminivirus sequences) that are attenuated (e.g., contain one or attenuating mutations). Methods of selecting attenuated virus strains are known in the art. Alternatively, attenuated strains may be routinely generated using standard methods of mutagenesis or genetic engineering techniques (such as site-directed mutagenesis).

Methods of Using the Virus Constructs of the Invention.

The present methods are useful in suppressing the production of any undesired gene product, e.g., sugars or other products contributing to the flavor, color or composition of a plant product. In addition, gene suppression may be used to vary the fatty acid distribution in plants such as rapeseed, Cuphea or jojoba, to delay the ripening of fruits and vegetables, to change the organoleptic, storage, packaging, picking, and/or processing properties of fruits and vegetables, to delay the flowering or senescing of cut flowers for bouquets, or to alter flower or fruit color. Exemplary genes that may be silenced include, but are not limited to, black phenol oxidase (browning in fruit), M-methylputrescine oxidase or putrescine N-methyl transferase (to reduce nicotine, e.g., in tobacco), polygalactouronase or cellulase (to delay ripening in fruits, e.g., tomatoes), ACC oxidase (to decrease ethylene production), 7-methylxanthine 3-methyl transferase (to reduce caffeine, e.g., in coffee, or to reduce theophylline, e.g., in tea), chalcone synthase, phenylalanine ammonia lyase, or dehydrokaempferol hydroxylases (to alter flower color, e.g, in ornamental flowers), cinnamoyl-CoA:NADPH reductase or cinnamoyl alcohol dehydrogenase (to reduce lignin content, e.g., in pine, fir and spruce), GL1 (to block trichome development to produce “hairless” leaves or fruit, e.g, peaches), cellulose (to decrease “woody” tissue, e.g, in asparagus), 1,3-1,4-glucan in barley (to reduce “cloudy” beer), Prup1 (a peach and apricot allergen), and other plant allergens (e.g, peanut and other nut allergens), the gene encoding the toxic lectin protein or alkaloid ricinine in castor beans, genes required for seed or pit production in fruit, and the like. In addition, systemic or tissue-specific suppression of a particular endogenous protein product may be desirable where the plant has been transformed to express a protein product of interest; suppression of endogenous proteins may lead to increased production of the transgene of interest.

Although the vectors of the present invention are typically not transmitted =through the germ-line, the present inventors have observed that the silencing effects produced according to the present invention may persist at least through several generations in cultured cells. Accordingly, the present invention may be used to produce long-term suppression without stable integration into the plant genome and without germ-line transmission.

The present invention may further be advantageously used to suppress genes in plants that reproduce via asexual reproduction (e.g, potatoes, cassava, poinsettias, bananas, grapevines, fruit trees, and the like). Methods of asexual reproduction are known in the art and include, but are not limited to, reproduction by grafting, cuttings, stolons, rhizomes, splitting of plants and bulbs, and apomixis. The present invention is particularly advantageous in plants that are asexually reproduced by grafting. Roses, bananas and plantains, grapes, and fruit trees (e.g., apple, orange, pear, peach, nectarine, plum, cherry, apricot and the like) are illustrative examples of plants that may be reproduced by grafting.

The host plant need not be one that is naturally susceptible to the virus from which the silencing construct is derived. Particle bombardment techniques, as described below, may be used to introduce a silencing construct into a cell, or group of cells, in a plant.

Improved methods of isolating and sequencing gene sequences have provided many isolated plant DNA segments of unknown function. Methods of determining the function of DNA segments have not kept pace with methods of isolating or determining the sequence of DNA segments. The present constructs and methods provide a means of rapidly and reproducibly screening plant DNA sequences of unknown function to determine their function in plant cells, tissue or intact plants, using episomally-mediated homology-dependent gene silencing. Such screening methods typically include the preparation of an episomal silencing construct containing one or more heterologous DNA sequences identical to or having substantial sequence similarity to an endogenous plant gene(s) (as described above); inoculating host plants or host plant tissue or cells with the silencing construct and, after a period of growth, comparing the inoculated host with an uninfected control plant or control plant tissue or cells. It will be appreciated by those skilled in the art that the “test” plant and “control” plant may be the same. For example, the same plant may be compared before and after inoculation. Alternatively, and preferably, different parts of the same plant (e.g., different leaves) may be used for the “test” and “control” treatments.

It is not necessary that the sequence of the target sequence or the sequence of the heterologous DNA carried by the episomal silencing construct be known. For example, nucleotide sequences from a library (e.g., a random library or a plant cDNA library, or any other library of nucleotide sequences of interest) may be cloned into the episomal silencing vector and introduced into a plant as described herein. Plants exhibiting phenotypic characteristics of interest may be identified, the sequence of the library clone(s) of interest determined, and the corresponding plant target gene(s) identified by standard techniques. Such “functional genomic” approaches may be employed to rapidly identify gene functions of interest.

Constructs based on geminivirus, nanovirus, badnavirus and caulimovirus genomes are particularly useful, as these viruses are known to infect a wide variety of agriculturally important crop plants. Characteristics for comparing test and control plants include growth characteristics, morphology, observable phenotype (including phenotypes observable with microscopic techniques), and biochemical composition. The differences between the test and control plants indicate the function of the silenced DNA sequence. The period of growth necessary for any differences in the treated and control plants to become apparent will vary depending on the host plants used and the function of the DNA being suppressed, as will be apparent to one skilled in the art. Such periods may range from several days, a week, two weeks, three weeks or four weeks, up to six weeks, eight weeks, three months, six months or more. Because the present method does not require tissue culture or selection to obtain alterations in gene expression, the methods can be adapted to automation for large-scale screening of anonymous sequences for function in plants.

As used herein, “screening” of a DNA segment does not imply that the function of the DNA segment will be positively identified in every case. As used herein, an “unidentified” plant gene or DNA segment is one whose functional role in the plant is unknown, even though the nucleotide sequence may be known.

As used herein a method of screening or identifying the “function” of an endogenous plant gene is not intended to indicate that the function or action of the gene (and the associated gene product) is necessarily identified at the cellular or molecular level. The term “function,” as used herein, also refers to a phenotypic feature of the plant (or plant cell or plant tissue) that is associated with silencing of the endogenous plant gene, which phenotypic feature provides information related to the function or biological activity of the plant gene and its gene product. For example, the present invention may be used to silence plant genes which result in a stunting of growth, loss of chlorophyll, reduced stress tolerance, and the like. These plant genes would be presumptively identified as having functions related to normal growth, chlorophyll production, stress tolerance, respectively.

The present invention also provides methods for rapidly and reproducibly screening portions of an isolated plant gene of known function, to identify those portions or fragments of genes that are effective in preventing or suppressing expression. Such screening methods will lead to refinements in current methods of gene suppression using sense and antisense DNA.

According to the present methods, the plant (or plant cell or tissue) may be co-inoculated with both the geminivirus A and B genomic components, alternatively, both genomic components may be present on a single binary vector. For example, as described hereinbelow, the plant may be co-inoculated with a geminivirus silencing vector comprising a geminivirus A component and an additional construct comprising a geminivirus B component that provides the movement proteins for the silencing vector (and which may further be a silencing vector as well). Conversely, the plant may be co-inoculated with a geminivirus silencing vector comprising a geminivirus B component and a construct comprising a geminivirus A component that provides replication functions.

As still a further alternative, the plant may be co-inoculated with vectors comprising the geminivirus replication and movement proteins.

Alternatively, in particular embodiments, the test cell or plant may be stably transformed to express particular geminivirus genes and then inoculated with a geminivirus silencing vector, as described above, comprising a heterologous DNA sequence(s), where the sequence(s) has substantial homology to an endogenous plant gene or a fragment thereof. For example, the plant cell or plant may be stably transformed with a geminivirus A component (alternatively, geminivirus AL1, AL2, and/or AL3 genes), and is then inoculated with a silencing vector comprising a geminivirus B genomic component (alternatively, the geminivirus BR1 and/or BL1 genes). In this manner, the stably incorporated replication genes from the A component will support the replication of the silencing vector comprising the B component (or B component genes).

Conversely, the plant cell or plant may be stably transformed with a geminivirus B component (alternatively, the BR1 and/or BL1 genes), and is inoculated with a silencing vector comprising a geminivirus A component (alternatively, geminivirus AL1, AL2, and/or AL3 genes to provide replication functions). According to this embodiment, the B component movement proteins expressed from the plant genome will enhance movement of the silencing vector comprising the A component (or A component genes).

As described above, the silencing vector may be phloem-limited or non-phloem limited. In particular embodiments, a phloem-limited silencing virus (e.g., a geminivirus phloem-limited silencing vector) is preferred. The inventors have made the surprising discovery that gene silencing in cells outside of the phloem may be achieved with phloem-limited vectors.

In other preferred embodiments, silencing is observed in plant cells outside of the phloem, e.g., in mesophyll cells, epidermis cells, cortical cells, parenchymal cells, guard cells, xylem cells, floral cells, fruit cells, seed coat cells, meristematic cells, apical cells, sclerenchyma cells, and/or colenchyma cells. Silencing is not typically observed in the embryo or other cells within the seed (other than seed coat cells).

In particular embodiments, the invention further finds use in methods of screening two or more endogenous plant genes for function, as described more fully hereinbelow. In addition, this embodiment may be employed to explore complex metabolic pathways, which because of compensatory interactions or multiple (e.g., redundant) branches necessitates silencing of more than one gene to disrupt the pathway.

The ease and convenience of the present invention further advantageously allows multiple genes to be silenced to carry out genetic studies similar to those used in other model systems, such as yeast. For example, multiple genes may be suppressed for studies of “synthetic enhancement”, “synthetic lethality” or “epistatic” studies. In general, “synthetic lethality” and “synthetic enhancement” studies permit the identification of combinations of two or more genes, which when co-suppressed, enhance the severity of the phenotype more than when any one of the genes is suppressed. “Epistatic” studies may be used to define biochemical pathways, by identifying genes that give qualitatively similar phenotypes upon suppression (e.g., the genes are in the same epistatic group).

In a still further preferred embodiment of multiple gene silencing, one or more of the silenced genes is a transgene introduced into the plant encoding a reporter protein (e.g., GFP, luciferase, β-glucuronidase, β-galactosidase) or any other endogenous plant marker protein that will give rise to a readily observable phenotype upon silencing (e.g., the su gene or other genes required for synthesis of chlorophyll or other plant pigments). Silencing of the reporter or marker gene is a convenient indicator to assess the presence, extent and/or spread of silencing of other genes by the silencing vector. Those skilled in the art will appreciate that the silencing of different genes by the nucleotide sequences carried by the silencing vectors of the present invention may not be completely co-extensive. The suitability of any particular reporter or marker gene as an indicator of suppression of any other plant gene may be readily determined by those skilled in the art.

The present invention advantageously provides methods of silencing one or more endogenous plant genes using silencing vectors which are preferably derived from DNA plant viruses, more preferably geminiviruses, as described above. The silencing vector may be derived from a geminivirus A component or B component, or both. In particular embodiments, a plant (or plant cell or tissue) is inoculated with one or more silencing vectors derived from a geminivirus A component and one or more silencing vectors derived from a geminivirus B component. For example, in an illustrative preferred embodiment, a plant is inoculated with a silencing vector comprising a geminivirus A component and another silencing vector comprising a geminivirus B component. The geminivirus genomic components may be from the same geminivirus or may be “pseudorecombinants” as described above.

In a further preferred embodiment, silencing vectors comprising the squash leaf curl virus genomic A or B components are used to achieve gene silencing in Arabidopsis, canola or other species of Brassicaceae (as described below).

In still a further preferred embodiment, silencing vectors comprising the bean golden mosaic virus genomic A and/or B component is used to achieve gene silencing in soybeans.

In another preferred embodiment, silencing vectors comprising the cotton leaf curl virus genomic A and/or B component is used to achieve gene silencing in cotton.

The present DNA episomal silencing system provides advantages over RNA viral vectors that are currently in use for testing gene function. Infection with RNA viruses requires that infectious transcripts be made in vitro, capped, and mechanically inoculated. Other “knock-out” systems in plants rely on chromosomal transformation, which can be time-consuming. Unlike RNA virus-derived vectors, foreign DNA is stably maintained in geminivirus vectors and cloned DNA isolated from E. coli can be used directly for inoculation of intact plants, e.g., by particle bombardment. Infectious DNAs can be easily generated from shuttle vector libraries containing large segments of cDNA sequence. The present inventors have shown that as little as about 50 base pairs of transcribed sequence can result in effective silencing, obviating the need for cloning full-length cDNAs. Promoter sequences have also been silenced by TGMV vectors, indicating that individual members of gene families can be selectively silenced where their promoters differ sufficiently from one another. Geminivirus and badnavirus vectors can be developed for different families of plants, thus allowing genes to be characterized directly in a species of interest. The present invention can also be used to identify single gene traits in a variety of species. Libraries of genes can be tested by subjecting plants to a screen for a single gene trait, such as pathogen resistance, and then looking for susceptible plants whose gene for resistance has been silenced. Current mutagenesis techniques require screening of segregating progeny, which can be time-consuming and is not feasible for many species that carry genes of interest.

The present invention further provides a method of silencing genes in intact plants, plant cells, and plant tissues using a mobile silencing vector, without the need to regenerate entire plants from individual cells. Silencing of active plant genes may be achieved with homologous fragments carried by a silencing vector in either the sense or anti-sense direction. Silencing may be achieved with small gene fragments (e.g., approximately 50 nucleotides or less), and may be achieved in the absence of detectable transcription or translation of the homologous sequence carried by the silencing vector. Moreover, the present inventors have observed gene silencing in cells with the inventive geminivirus silencing vectors in the absence of viral replication within the cell. While not wishing to be held to a single theory of the invention, it appears that the silencing signal is diffusible in nature and may extend well beyond the cells in which the geminivirus replicates. For example, when TGMV vectors were inoculated by bombardment into plants, areas of hundreds of cells were silenced within 3-5 days, whereas the virus only replicated in 1-2 cells (Kjemtrup et al., (1998) Plant J. 14:91).

CbLCV Silencing Vectors.

In particular preferred embodiments of the invention, the silencing vector is derived from the Cabbage Leaf Curl Virus (CbLCV). CbLCV silencing vectors may be used with any suitable plant (or plant cell or tissue), preferably a species of Brassicaceae (as set forth in more detail below), more preferably Arabidopsis. In other preferred embodiments, the plant is a tobacco plant. Moreover, the inventive CbLCV vectors may be used in the silencing and screening methods set forth herein.

The present inventors have made the discovery, that unlike most geminiviruses, CbLCV is non-phloem limited. Silencing may be achieved with CbLCV silencing vectors in cells outside of the phloem, e.g., in mesophyll cells, epidermis cells, cortical cells, parenchymal cells, guard cells, xylem cells, floral cells, fruit cells, meristematic cells, seed coat cells, apical cells, sclerenchyma cells, and colenchyma cells.

CbLCV silencing vectors are as described above with respect to geminivirus silencing vectors. In one preferred embodiment, the silencing vector comprises a CbLCV genomic component, which comprises one or more heterologous DNA sequences, each of the heterologous DNA sequences having substantial sequence similarity to an endogenous plant gene or a fragment thereof. The CbLCV genomic component may be the A component or the B component. As a further alternative, the silencing vector may be a binary vector that comprises sequences from both the CbLCV A component and the B component. Those skilled in the art will appreciate that the CbLCV silencing vectors may comprise “hybrid” or “pseudorecombinant” geminivirus sequences, as described above.

In other preferred embodiments, the CbLCV genomic component is attenuated, so that pathological effects in the plant (or plant cell or tissue) inoculated with the CbLCV silencing vector are reduced as compared with the effects observed with a wild-type or non-attenuated CbLCV silencing vector.

In other preferred embodiments, the silencing vector comprises a CbLCV origin of replication, CbLCV genes necessary for replication of the vector in a plant cell (e.g., AL1, AL3 and/or AL2 genes, preferably AL1 and AL3), and one or more heterologous DNA sequences, each of the heterologous DNA sequences having substantial sequence similarity to an endogenous plant gene or a fragment thereof. The silencing vector may further comprise geminivirus (preferably CbLCV) sequences required for movement (e.g., BR1 and/or BL1 genes).

In a still further preferred embodiment, the silencing vector comprises a CbLCV origin or replication, the CbLCV movement sequences (e.g., BR1 and/or BL1 genes, preferably both), and one or more heterologous DNA sequences, each of the heterologous DNA sequences having substantial sequence similarity to an endogenous plant gene or a fragment thereof. The silencing vector may further comprise geminivirus (preferably CbLCV) sequences required for replication (e.g., AL1, AL2 and/or AL3 genes).

In particular preferred embodiments, the CbLCV genomic component is pseudorecombinant between CbLCV and squash leaf curl virus (see, e.g., Hill et al., (1998) Virology 250:283). Such pseudorecombinants may have reduced pathological effects on host plants.

Subject Plants.

Plants that may be employed in practicing the present invention include any plant (angiosperm or gymnosperm; monocot or dicot) in which DNA constructs according to the present invention can replicate and, where systemic silencing is desired, where movement of the DNA construct or a silencing factor occurs. Particularly preferred are those plants susceptible to infection by plant geminiviruses. As used herein, “susceptible to infection” includes plants that are naturally infected by geminiviruses in the wild, plants that can be mechanically inoculated with the DNA construct, or that can be inoculated by methods other than mechanical inoculation (such as by Agrobacterium inoculation). “Susceptible to infection” refers to plants in which the DNA construct is able to replicate within the inoculated plant cell.

Exemplary plants include, but are not limited to corn (Zea mays), canola (Brassica napus, Brassica rapa ssp.), alfalfa (Medicago saliva), rice (Oryza sativa), rape (Brassica napus), rye (Secale cereale), sorghum (Sorghum bicolor, Sorghum vulgare), sunflower (Helianthus annus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris). duckweed (Lemna), oats, barley, vegetables, ornamentals, conifers, and turfgrasses (e.g., for ornamental, recreational or forage purposes).

Vegetables include Solanaceous species (e.g., tomatoes; Lycopersicon esculentum), lettuce (e.g., Lactuea sativa), carrots (Caucus carota), cauliflower (Brassica oleracea), celery (Apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysiagrasses, bentgrasses, fescue grasses, bluegrasses, St. Augustinegrasses, bermudagrasses, bufallograsses, ryegrasses, and orchardgrasses.

Also included are plants that serve primarily as laboratory models, e.g., Arabidopsis.

Preferred plants for use in the present methods include (but are not limited to) legumes, solanaceous species (e.g., tomatoes), leafy vegetables such as lettuce and cabbage, turfgrasses, and crop plants (e.g., tobacco, wheat, sorghum, barley, rye, rice, corn, cotton, cassava, and the like), and laboratory plants (e.g., Arabidopsis).

Also preferred are members of the Brassicaceae family, which include but are not limited to: Turritis glabra, Thlaspi rotundifolium, Thlaspi arvense, Teesdalea nudicaulis, Streptanthus cordatus, Stanleya pinnata, Sisymbrium sophia, Sisymbrium officinale, Sisymbrium loeselii, Sinapis arvensis, Sinapis alba, Raphanus sativus, Raphanus raphanistrum, Radicula palustris, Radicula nasturtium aquaticum, Physaria chambersii, Nerisyrenia camporum, Neobeckia aquatica, Lunaria rediviva, Lunaria annua, Lobularia maritima, Lesquerella sp., Lesquerella rubicundula, Lesquerella densiflora, Lesquerella argyraea, Lepidium virginicum, Lepidium ruderale, Lepidium flavum, Isatis tinctoria, Hesperis matronalis, Erysimum capitatum, Erysimum asperum, Draba verna, Draba rupestris, Draba alpina, Descurainia pinnata, Dentaria laciniata, Dentaria bulbifera, Crambe maritima, Cochlearia officinalis, Cardamine pratensis, Cardamine bellidifolia, Capsella bursapastoris, Camelina sativa, Cakile maritima, Bunias orientalis, Brassica ruvo, Brassica rapa subsp. Chinensis, Brassica oleracea var. gongylo, Brassica oleracea, Brassica oleracea var. sabellica, Brassica oleracea var. gongylodes, Brassica nigra, Brassica napus var. napus, Brassica napus, Brassica napus var. napobrassica, Brassicajuncea, Biscutella laevigata, Berteroa incana, Barbaraea lyrata, Armoracia rusticana, Arabis pumila, Arabis petiolaris, Arabis alpina, Arabidopsis thaliana, Alliaria petiolata, and Alliaria officinalis.

Transformation Methods.

Plants can be transformed according to the present invention using any suitable method known in the art. Intact plants, plant tissue, explants, meristematic tissue, protoplasts, callus tissue, cultured cells, and the like may be used for transformation depending on the plant species and the method employed. In a preferred embodiment, intact plants are inoculated using microprojectiles carrying a geminivirus silencing vector according to the present invention. The site of inoculation will be apparent to one skilled in the art; leaf tissue is one example of a suitable site of inoculation. In preferred embodiments, intact plant tissues or plants are inoculated, without the need for regeneration of plants.

Exemplary transformation methods include biological methods using viruses and Agrobacterium, physicochemical methods such as electroporation, polyethylene glycol, ballistic bombardment, microinjection, and the like. Transformation by ballistic bombardment is preferred.

In one form of direct transformation, the vector is microinjected directly into plant cells by use of micropipettes to mechanically transfer the recombinant DNA (Crossway, Mol. Gen. Genetics 202:, 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl. Acad. Sci. USA 79, 1859 (1982)).

DNA may also be introduced into the plant cells by electroporation (Fromm et al., Proc. Natl. Acad. Sci. USA 82, 5824 (1985)). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide and regenerate. One advantage of electroporation is that large pieces of DNA, including artificial chromosomes, can be transformed by this method.

Viruses include RNA and DNA viruses, with DNA viruses (e.g., geminiviruses, badnaviruses, nanoviruses and caulimoviruses) being preferred, and geminiviruses being more preferred.

Ballistic transformation typically comprises the steps of: (a) providing a plant tissue as a target; (b) propelling a microprojectile carrying the heterologous nucleotide sequence at the plant tissue at a velocity sufficient to pierce the walls of the cells within the tissue and to deposit the nucleotide sequence within a cell of the tissue to thereby provide a transformed tissue. In particular preferred embodiments of the invention, the method further includes the step of culturing the transformed tissue with a selection agent. In a more preferred embodiment, the selection step is followed by the step of regenerating transformed plants from the transformed tissue. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et al., Proc. Natl. Acad. Sci. USA 85, 4305 (1988)), soybean callus (Christou et al., Plant Physiol 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target tissue is positioned in the bombardment chamber so that a microprojectile(s) propelled through the bore hole penetrates the cell walls of the cells in the target tissue and deposit the nucleotide sequence of interest carried thereon in the cells of the target tissue. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic transformation is achieved without use of microprojectiles. For example, an aqueous solution containing the nucleotide sequence of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target tissue (or both).

It is currently preferred to carry the nucleotide sequence on a microprojectile. The microprojectile may be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Metallic particles are currently preferred. Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their DNA carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes, preferably Agrobacterium tumefaciens. Agrobacterium-mediated gene transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Gene transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye (de la Pena et al., Nature 325, 274 (1987)), maize (Rhodes et al., Science 240, 204 (1988)), and rice (Shimamoto et al., Nature 338, 274 (1989)).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrum L., and poplar. U.S. Pat. No. 5,777,200 to Ryals et al. As described by U.S. Pat. No. 5, 773,693 to Burgess et al., it is preferable to use a disarmed A. tumefaciens strain (as described below), however, the wild-type A. rhizogenes may be employed. An illustrative strain of A. rhizogenes is strain 15834.

The Agrobacterium strain utilized in the methods of the present invention is modified to contain the nucleotide sequences to be transferred to the plant. The nucleotide sequence to be transferred is incorporated into the T-region and is typically flanked by at least one T-DNA border sequence, preferably two T-DNA border sequences. A variety of Agrobacterium strains are known in the art particularly, and can be used in the methods of the invention. See, e.g., Hooykaas, Plant Mol. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996), the disclosures of which are incorporated herein by reference.

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J 3, 1681 (1984), and the non-oncogenic Ti plasmid pGV2850 described by Zambryski et al., EMBOJ 2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous nucleotide sequence of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662, herein incorporated by reference. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J. Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616, herein incorporated by reference) and pTOK233 (Komari, Plant Cell Reports 9, 303 (1990); Ishida et al., Nature Biotechnology 14, 745 (1996); herein incorporated by reference). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

Plant cells may be transformed with Agrobacteria by any means known in the art, e.g., by co-cultivation with cultured isolated protoplasts, or transformation of intact cells or tissues. The first requires an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

In plants stably transformed by Agrobacteria-mediated transformation, the nucleotide sequence of interest is incorporated into the plant genome, typically flanked by at least one T-DNA border sequence. Preferably, the nucleotide sequence of interest is flanked by two T-DNA border sequences.

Plant cells, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). It is known that practically all plants can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

A large number of plants have been shown capable of regeneration from transformed individual cells to obtain transgenic whole plants.

The regenerated plants selected from those listed are transferred to standard soil conditions and cultivated in a conventional manner. The plants are grown and harvested using conventional procedures.

The particular conditions for transformation, selection and regeneration may be optimized by those of skill in the art. Factors that affect the efficiency of transformation include the species of plant, the tissue infected, composition of the media for tissue culture, selectable marker genes, the length of any of the above-described step, kinds of vectors, and light/dark conditions. Therefore, these and other factors may be varied to determine what is an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

The examples, which follow, are set forth to illustrate the present invention, and are not to be construed as limiting thereof. In the following examples, bp means base pair, cDNA means copy DNA, μg means microgram, ORF means open reading frame, and min means minute.

### EXAMPLE 1

### Materials and Methods: Vector Construction

This example describes the generation of recombinant A and B vectors of TGMV and CbLCV for introduction into plants.

TGMV Vectors.

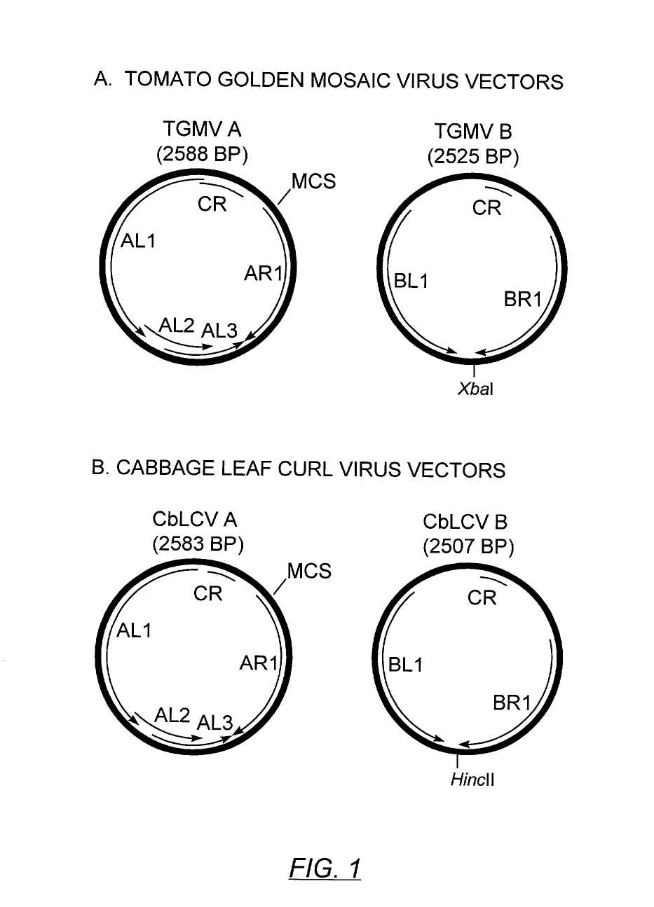

TGMV A-derived vectors were constructed using the pMON1655 plasmid, a pUC-based plasmid with 1.5 tandem copies of TGMV A containing the AR1 coding sequence replaced by a short polylinker, and retaining the AR1 promoter and terminator sequences (FIG. 1A). pLVN44 is a full-length 1392 bp cDNA of the nucleotide-binding subunit of magnesium chelatase (su) isolated from Nicotiana tabacum cv. SR1 (Nguyen, Transposon tagging and isolation of the sulfur gene in tobacco, Ph.D. Thesis, North Carolina State University (1995)). Magnesium chelatase is a multi-subunit protein that catalyzes the insertion of magnesium into protoporphyrin IX (Jensen et al., Molec. Gene Genetics 250:283 (1996)). In tobacco, a mutated allele (Su) of one subunit causes the phenotype known as ‘sulfur’. Nicotiana tabacum plants homozygous for this allele are yellow (Su/Su), and heterozygous plants are yellow-green (Su/su).

To generate a su::gfp fusion gene, a 361 bp fragment, corresponding to nt 627 to 986, of the su cDNA was amplified from pLVN44 with a 5′ PCR primer containing an BglII site and a 3′ PCR primer containing an XbaI site. A 388 bp fragment, corresponding to nt 130 to 518, of the gfp gene was amplified from mGFP5 template DNA (Haselhoff et al. (1997) Proc. Natl. Acad. Sci. USA 18:2122-2127) with a 5′ PCR primer containing an XbaI site and a 3′ PCR primer containing an Acc65I. The pMON1655 plasmid, containing 1.5 copy of TGMV, was restricted with Acc651 and Bgl/III. The gfp and su fragments were three-way ligated into pMON1655 to generate pMT001.

TGMV B vector pTG1.3BXSR (Schaffer et al. (1995) Virology 214:330-338) (FIG. 1A) containing 1.3 tandem, direct repeats of the TGMV B component, was used as a vector for inserting foreign DNA into the B component. The B component encodes two movement proteins, BL1 and BR1. A unique XbaI site was introduced 20 bp downstream of the BR1 ORF for insertion of foreign sequences. This site occurred before the putative BR1 polyadenylation site, allowing for co-transcription of the BR1 gene and the foreign DNA. Post-inoculation into plants, the E. coli portion of pTG1.3BXSR-derived vectors is excised, resulting in an episomal plasmid.

A plasmid harboring a su::gfp chimeric fragment and plasmids harboring various sizes of the su gene alone were prepared for introduction into plants. Primers containing NheI sites were used to amplify a su::gfp fragment from pMT001 with 58 bp of homology to the su gene and 72-bp of homology to GFP, which was ligated into the Xbal site of pTG1.3BXSR to create plasmid TG1.3B::GFP-su (Table 1).

To construct TGMV B plasmids harboring different-sized fragments of the 1398 bp N. tabacum su cDNA, pLVN44, harboring the su cDNA, was restricted with three different enzyme combinations and blunt-end cloned into the blunt-ended XbaI site of pTG1.3BXSR. The resulting plasmids, TGMV B::154su, NBsu1455, and NB935, are outlined in Table 1. A 154-bp fragment from the 5′ end of the N. benthamiana su gene was amplified using the following primers containing an XbaI site, 5′ gatctagaGGGAGGAAGTTTTATGGAGG 3∝ (SEQ ID NO:1) and 5′ gatctagaTAGCTGCAAATGGATACACCG 3′ (SEQ ID NO:2).

Proliferating cell nuclear antigen (PCNA) is required for DNA replication (Nagar et al., Plant Cell 7:705 (1995)) and acts as an accessory factor for DNA polymerase delta. The inventors inserted a 180-bp fragment of the pcna gene either (1) singly, (2) as a tandem duplication, or (3) as a 122-bp fragment of the pcna gene into the TGMV B component to determine if a phenotype could be observed.

TABLE 1

| | |
|---|---|
| Name | Heterologous insert |

TMGV A-Derived Constructs

| | |
|---|---|
| TGMV A::gfp | Full-length GFP fragment replacing AR1 locus |
| TGMV A::51su | 51-bp su fragment replacing the AR1 locus |
| TGMV A::92su | 92-bp su fragment replacing the AR1 locus |
| TGMV A::790 | 786-bp fragment of su gene, sense orientation, |
| | replacing the AR1 locus |
| MT0001 | 749-bp fragment with 361-bp of homology to the |
| | su gene and 388-bp of homology to gfp, sense |
| | orientation |
| TGMV A::650PCNA | 650-bp PCNA fragment inserted into A |
| | component |

TGMV B-Derived Constructs

| | |
|---|---|
| pTG1.3B::GFP-su | 140-bp fragment with 58-bp of homology to the |
| | su gene and 72-bp of homology to gfp, sense |
| | orientation |
| TGMV B::92su | 92-bp fragment, corresponding to nt 781-873 of |
| | the su cDNA, antisense orientation |
| TGMV B::154su | 154-bp SacI/EcoRV fragment, corresponding to |
| | nt 785-939 of the su cDNA, both orientations |
| | have heen tested with similar results |
| NBsu1455 | 479-bp SacI/SacI fragment, corresponding to nt |
| | 936-1415 of the su cDNA, antisense orientation |
| NB935 | 935-bp SacI/Acc65I fragment, corresponding to nt |
| | 0-935 of the su cDNA, 18 nt of 5′ untranslated |
| | region and 917 nt ORF, antisense orientation |
| TGMV B::122PCNA | 122-bp PCNA fragment inserted into B |
| | component |
| TGMV B::180PCNA | 180-bp PCNA fragment inserted into B |
| | component |
| TGMV | Tandem direct repeat of 180-bp PCNA |
| B::180PCNAtr | fragment inserted |
| | into B component |

CbLCV Vectors.

A plasmid containing the CbLCV A component, with two copies of the common region and two copies of AL1, was constructed by ligating a 1.6-kb EcoRI/HindII fragment from the original clone (provided by Dr. Ernie Hiebert) into similar sites of the polylinker of pBS. The clone from Dr. Hiebert was then used a second time to provide a 1.8-kb fragment by digestion with AatII and EcoRI. First the plasmid was digested with AatII and the ends filled-in to make them blunt (compatible with SmaI). The vector was then digested with EcoRI to produce one sticky and one blunt end. This was ligated to the EcoRI/SmaI sites of the construct containing the 1.6-kb EcoRI/HindII. This construct is called pCLCVA.003 and has a 1.3 tandem direct repeat containing two copies of the common region. The A-derived vector has the AR1 coding sequence replaced by a short polylinker, but retains the AR1 promoter and terminator sequences (FIG. 1B). The fragment of Ch-42 used for silencing in CbLCV was isolated from a PCR fragment generated with primers CH42_{—}1₁R (5′ ACT GTT AGA TCt TTA GTT GAT CTG 3′ (SEQ ID NO:3)) and CH42_{—}1_L (5′ AAT CCC TTC TCT aga AAC CGT AAT CCA ACC 3′ (SEQ ID NO:4)). These primers anneal to the open reading frame of Ch-42 at positions 382-405 and 733-762 respectively. Restriction sites were introduced into CH42_R 1 (BglII) and CH42_L_{—}1 (XbaI) by introducing mismatches into the primers. The engineered restriction sites are indicated by bold underlined text in the primer sequences, while the mismatches are indicated by lower case letters. The PCR fragment was digested with BglII and XbaI and the resulting fragment ligated into BglII/XbaI digested CbLCV vector (containing the multi-cloning site and no coat protein gene). The resulting clone produces an RNA (from the CbLCV protein promoter) which has a fragment of 353 nucleotides which is completely homologous to the Ch-42 gene (position 394-747 in the Ch-42 open reading frame).

To make a 1.5 copy tandem, direct-repeat of the B component, CLCVB/pGEMEX-1 (provided by Dr. Ernie Hiebert) was digested with EcoRI/EcoRV. A 1.4-kb fragment was isolated and ligated into EcoRI/SmaI sites of pBluescript SK+II and named pCLCVB.001. CLCVB/pGEMEX-1 was digested a second time with EcoRI releasing one unit-length copy of the viral B genome. This copy was ligated into an EcoRI site of pCpCLCVB.001 to make cCpClCVB.002 (FIG. 1B). Experiments described herein for CbLCV-mediated transformation use plasmids derived from pCpClCVB.002.

An additional modification was performed to preserve the BR1 stop codon in the CbLCV B vector. pCpClCVB.002 was subsequently digested with Acc651 and SalI, blunt-ended with the Klenow fragment and religated to generate pCpC1CVB.003. This vector was then modified by the addition of a double-stranded linker sequence corresponding to AAGGTACCTT (SEQ ID NO:5) which was blunt-end ligated into pCpCaLCVB.003 digested with at HincII. The resulting vector was sequenced to confirm the Acc651 site and named pNMCLCVB. The additional AA at the 5′ end of the linker in SEQ ID NO:5 is needed to preserve the stop codon (TAA) for the BR1 gene. Cloning directly into HincII does not retain the stop codon which reduces silencing.

TABLE 2

| | |
|---|---|
| Name | Heterologous insert |

CbLCV A-Derived Constructs

| | |
|---|---|
| CbLCVA::su | 784 bp fragment, corresponding to nt 1-786 |
| | of the su cDNA, sense orientation. |
| CbLCVA::CH42 | 364-bp fragment, corresponding to nt |
| | 1209-1560 of the Ch-42 cDNA, antisense |
| | orientation. |
| CbLCV A::CH42- | 344-bp fragment of Ch-42 cDNA and 394 bp |
| GFP | fragment GFP of gfp gene |
| CbLCVA::GFP | 383 bp fragment, corresponding to nt 115-498 |
| | of the gfp cDNA |
| CbLCV A (-AR1) | Deletion of AR1 coat protein gene |

CbLCV B-Derived Constructs

| | |
|---|---|
| CbLCV B::su | 154-bp SacI/EcoRV fragment, corresponding |
| | to nt 785-939 of the su cDNA, both |
| | orientations were tested |
| CbLCV B::CH42 | 144-bp fragment, corresponding to nt |
| | 1209-1353 of the Ch-42 cDNA, sense orientation |

### EXAMPLE 2

### Materials and Methods: Plant Transformation

This example describes the introduction of the recombinant TGMV and CbLCV-derived plasmids into plants and protocols employed for in situ hybridization to localize TGMV and CbLCV DNA in transformed plant tissues.

Wild type N. benthamiana or N. benthamiana, transgenic for the green fluorescent protein (gfp) driven by the constitutive 35S CaMV promoter, were used in all experiments. The BIOLISTIC® Particle Delivery System (Bio-Rad, Hercules, Calif., USA) was used to infect three-week-old Nicotiana seedlings in two-inch plastic pots. Individual seedlings were bombarded with microprojectiles coated with equal amounts (5 μg each) of various combinations of TGMV A and B plasmid DNAs as disclosed in Nagar et al. (Plant Cell 7:705 (1995). The TGMV B construct alone was used as a negative control, as it cannot replicate without the TGMV A component. Total DNA was isolated from plants and 5 μg from each plant was separated by electrophoresis and blotted as described (Kjemtrup et al. (1998) Plant J. 15:91-100). Digoxigenin-labeled probes were prepared using a Dig-High Prime kit from Roche Biochemicals (Indianapolis, Ind.) followed by chemiluminescent detection. PCR analysis of insert size in the B component vector was done using the primers: BR1 5′ GTCGGATATTGTGTCAAAGG 3′ (SEQ ID NO:6) and BL1 5′ TCTACTATTGGGCTAACAGG 3′ (SEQ ID NO:7) in a 50 μl reaction with 5 ng template DNA.

Similarly, wild type A. thaliana or A. thaliana, transgenic for the green fluorescent protein (gfp) driven by the constitutive 35S CaMV promoter, were used in all CbLCV-A. thaliana experiments. The BIOLISTIC® Particle Delivery System (Bio-Rad, Hercules, Calif., USA) was used to transform Arabidopsis plants. Two different stages of plant development were used in the following experiments. (1) Individual seedlings grown in four-inch plastic pots under short days to promote vegetative growth and well-developed rosettes. (2) Four seedlings on 2.5-cm plates which were transplanted 2 days post-bombardment and then grown under short days. In each experiment, plants were bombarded with microprojectiles coated with equal amounts (5 μg each) of various combinations of CbLCV A and B plasmid DNAs. The CbLCV B construct alone was used as a negative control, as it cannot replicate without the CbLCV A component.

### EXAMPLE 3

### Materials and Methods: in situ Hybridization Analysis

TGMV.

Digoxigenin-labeled probes were prepared using digoxigenin d-UTP from Roche Biochemical. A 281-bp sequence from the AL1 gene of TGMV was labeled using PCR. Tissues were fixed, embedded in agarose, and vibratome sectioned. Sections were incubated in 1 ng/μl digoxigenin probe overnight at 37° C., followed by incubation with anti-digoxigenin conjugated alkaline phosphatase and detection in nitoblue tetrazolium/5-bromo-4-chloro-indolyl-phosphate. Both TGMV-silenced tissue and wild type TGMV tissue were incubated in substrate for 1 h. Immunolocalization of PCNA used monoclonal antibody PC 10 (Santa Cruz Biotechnology, Inc., Santa Cruz, Calif.) detected using an avidin biotin horseradish peroxidase conjugate with amino-ethylcarbazole substrate (Zymed Laboratories, San Francisco, Calif.). (Nagar et al. (1995) Plant Cell 7:705-719).

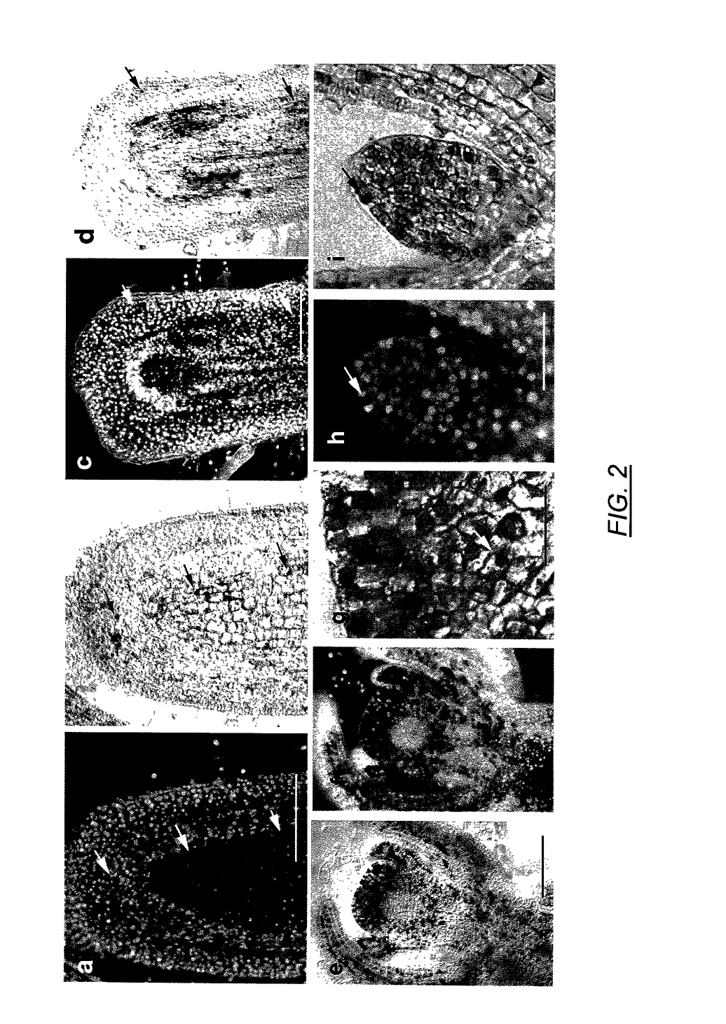

Meristem culture is a common method for obtaining plants that lack viruses, presumably because viruses are unable to access meristematic tissues. Geminiviruses are not seed transmitted, and although viral DNA is found in the seed coat, embryos are not infected (Sudarshana et al. (1998) Mol. Plant-Microbe Interact. 11:277-291). In situ hybridization studies demonstrated that plant meristematic areas lack geminivirus DNA (Horns and Jeske (1991) Virology 181:580-588; Lucy et al. (1996) Mol. Plant Microbe Interact. 9:22-31). To determine if silencing of PCNA occurred in the meristem, or if restriction of PCNA expression in subtending tissues negatively impacted development, immunolocalization of PCNA in infected meristems was performed. Whereas meristems were distinct and easy to dissect in TGMV A::790su/B and wild type TGMV-infected plants, plants infected with pTG1.3B::GFP-su or TGMV A::790su/TGMV B::122PCNA contained numerous leaves, little internode expansion, and aberrant and reduced meristems. Some plants appeared to lack a meristem. Examination of sections from plants that did contain a meristematic structure demonstrated that PCNA expression was greatly reduced in the terminal portions of the apex. Meristems silenced for PCNA lacked detectable leaf primordia (FIG. 2). There appeared to be a zone of cortical cells extending into the meristem subtended by a layer of cells that contained higher levels of PCNA. Isolated cells or groups of cells in the “cortical zone” showed PCNA staining (FIG. 2, panel B and FIG. 2, panel D, arrows). However, large sectors of the meristem showed very little expression. The meristem remained symmetrical, suggesting that cessation of PCNA expression affected development as well as DNA replication. Together, these observations demonstrated that silencing of the endogenous PCNA gene occurred in meristematic tissue.

CbLCV.

For localization of CbLCV, a fragment from CbLCV was labeled using PCR and plant tissues were fixed, embedded in agarose, and vibratome-sectioned using standard procedures. Tissues transformed with recombinant CbLCV were incubated in substrate overnight at 37° C. whereas tissues transformed with wild type CbLCV were incubated for 15-30 min at 37° C.

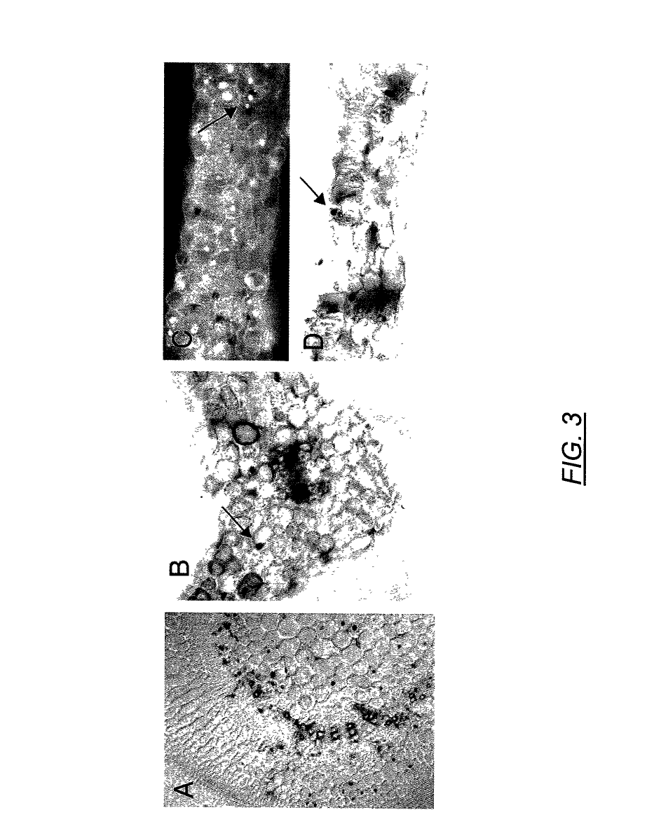

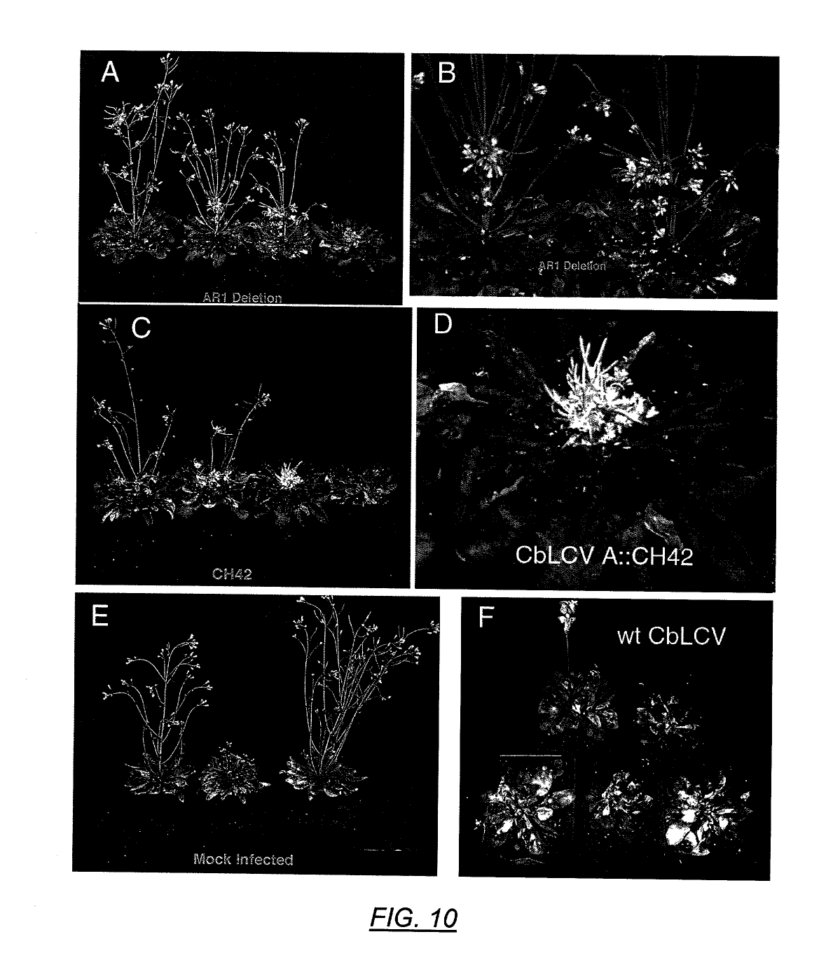

In situ hybridizations with digoxigenin-labeled CbLCV DNA showed that CbLCV was not phloem-limited in N. benthamiana stems or leaves (FIG. 3, panels A and B, respectively). CbLCV-infected nuclei were observed outside of vascular tissue. Arabidopsis plants transformed with CbLCV showed severe symptoms such as necrosis, chlorosis, and leaf curling (FIG. 10, panel F). Influorescences were curved, stunted, and flower formation was greatly reduced. In situ hybridizations with a digoxigenin-labeled CbLCV DNA probe demonstrated that CbLCV was not phloem-limited in Arabidopsis (FIG. 3, panel D).

### EXAMPLE 4

### Viral DNA Accumulation is Reduced in Silenced Tissue

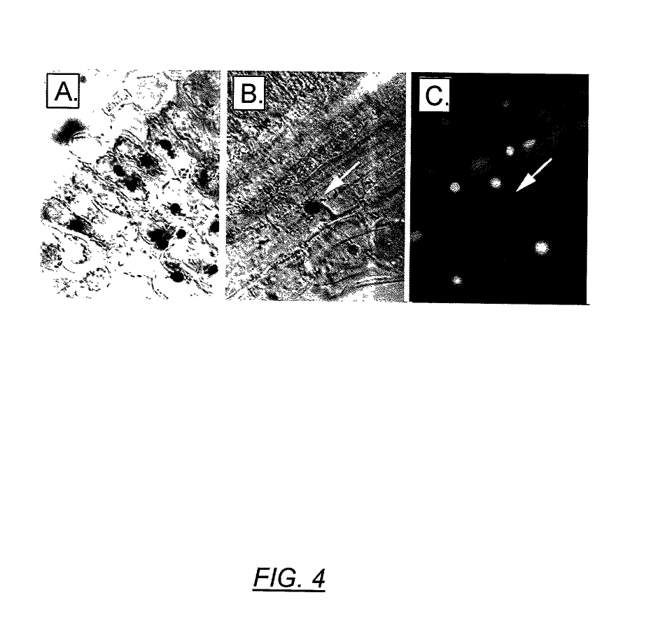

Attenuation of symptoms in TGMV-silenced plants suggested that geminivirus-induced silencing could be accomplished with only minor alterations in host gene expression and physiology. Previous results showed that viral DNA accumulation was reduced in TGMV::su silenced plants (Kjemtrup et al. (1998) Plant J. 14:91-100) but did not address the cellular basis of the reduction. To better understand viral infection of silenced tissue, in situ hybridization was used to determine the pattern of viral DNA accumulation in plants systemically infected with TGMV A::790su/B. Viral DNA was detected in isolated cells of green, non-silenced tissue (data not shown) and very rarely in silenced tissue (FIG. 4, panel B and panel C). Large areas of silenced tissue contained no detectable viral DNA. This pattern contrasted strongly with that of wild type TGMV, which accumulated in clusters of adjacent cells (FIG. 4, panel A). Viral DNA may still be present at low copy numbers in some cells lacking detectable digoxigenin signal, but the cellular pattern of productive viral replication was clearly different in silenced tissue compared to wild type TGMV-infected tissue.

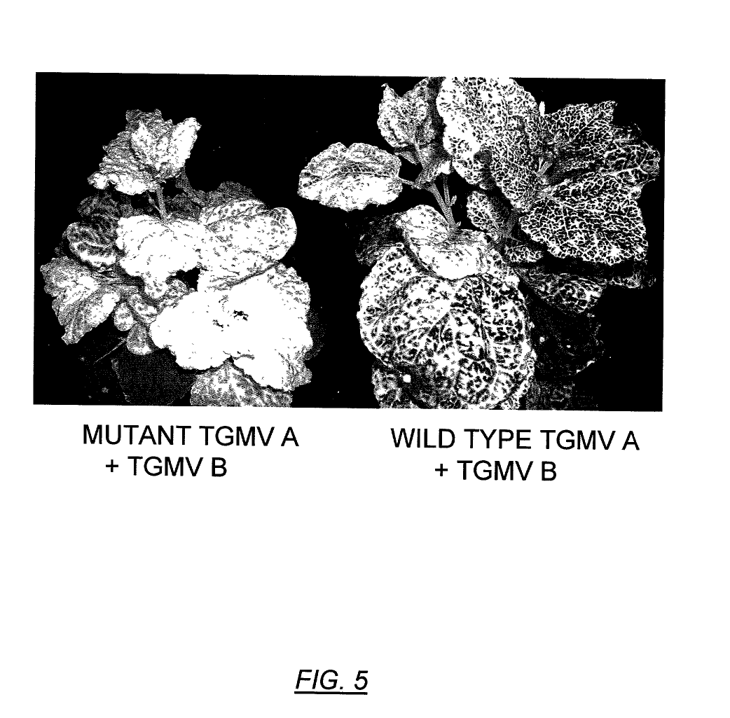

Although systemic silencing has been readily shown using transgenes (Kjemtrup et al. (1998) Plant J. 14:91-100; Ruiz et al. (1998) Plant Cell 10:937-946; Voinnet and Baulcombe (1997) Nature 389:553-553; Voinnet et al. (1998) Cell 95:177-187), only limited movement of a putative silencing signal has been demonstrated for endogenous genes. Our in situ results for su (FIG. 4, panel B and panel C) suggested that silencing of su could occur in cells that lacked detectable levels of viral DNA. To further test this idea, we used a mutant TGMV A component that restricts viral DNA replication to vascular tissue in N. benthamiana (Kong et al. (2000 EMBO J. 19:3485-3495). Plants inoculated with the mutant TGMV A component and TGMV B::154su showed extensive silencing (FIG. 5). Unlike the mutant TGMV (Kong et al. (2000 EMBO J. 19:3485-3495), the silencing signal was not restricted to the phloem.

### EXAMPLE 5

### Size Limitation of Foreign DNA inserted into Geminivirus Vectors

TGMV in N. Benthamiana.

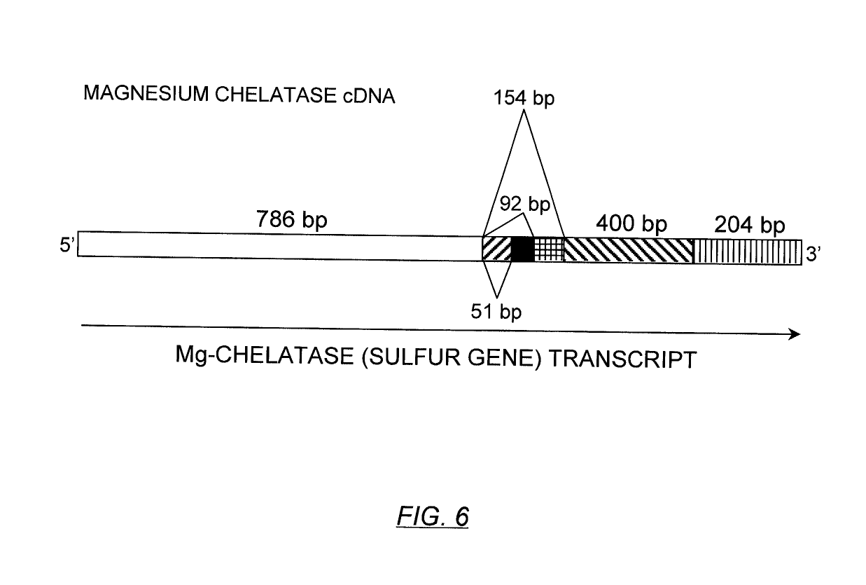

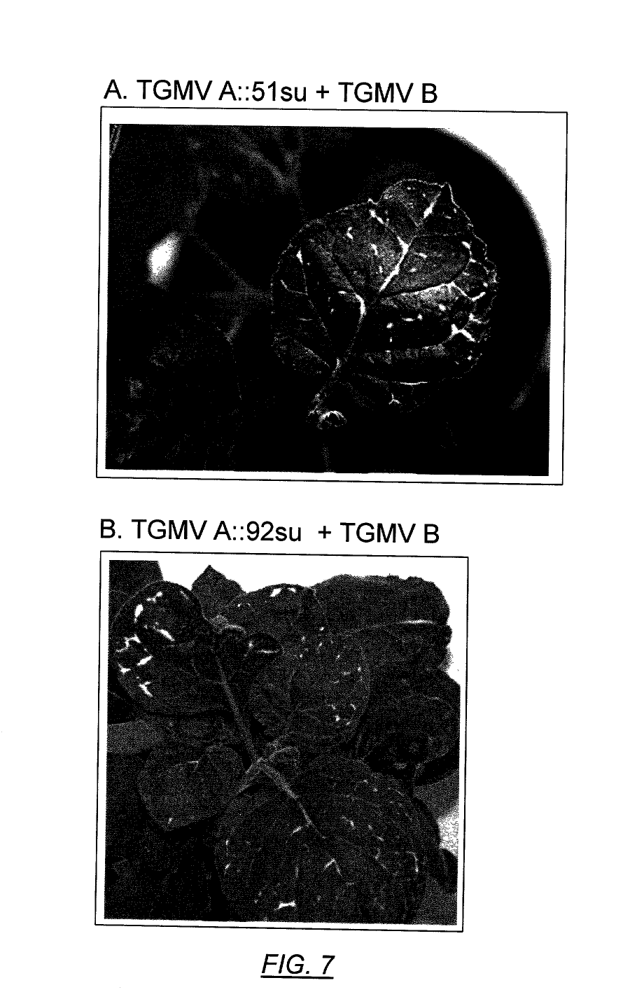

To determine the shortest sequence necessary to induce silencing, various-sized fragments from the middle of the su gene (FIG. 6) were cloned into the TGMV A vector and co-bombarded into plants with TGMV B DNA. Minimal silencing was seen when a 51-bp fragment of su (TGMV A::51 su; see Table 1 for nomenclature) was bombarded into plants. Although viral transcription of larger su fragments caused circular yellow spots on bombarded leaves 5 days post-inoculation, no silencing was observed initially in tissue bombarded with TGMV A::51su/B. Instead, variegation was confined to veins in new growth and only some plants showed silencing in three experiments (FIG. 7, panel A). In contrast, TGVM A::92su/B, containing 41-bp of additional su sequence, consistently caused silencing in every inoculated plant (FIG. 7, panel B) and produced yellow spots on target tissue. These results demonstrated that 51-bp of homologous sequence is near the lower limit for induction of endogenous gene silencing of su by TGMV A vectors, while a 92-bp fragment was highly effective for silencing.

One reason that the TGMV B component may be more effective for silencing is that the TGMV B component vector is co-bombarded with wild type TGMV A, retaining all TGMV genes including the coat protein gene. To test this, plants were inoculated with a su fragment in the TGMV A vector and in the TGMV B vector. Inoculation of TGMV A with a 786-bp fragment of the su gene, replacing the AR1 gene, and with wild type TGMV B resulted in variegation of the inoculated leaves (data not shown). Inoculation with wild type TGMV A and a TGMV B vector, containing a 154-bp su gene fragment (A/TGMV B::154su), resulted in white plants (data not shown). If the coat protein gene were required for extensive spread of the silencing signal, plants inoculated with TGMV A containing the 786-bp fragment of the su gene and TGMV B::154su should have resulted in a variegated phenotype. However, the extent of silencing with 786-bp su gene fragment in conjunction with TGMV B::154su was similar to TGMV B::154su inoculated with wild type A (retaining the AR1 gene). It was concluded that the AR1 gene is not required for extensive spread of the silencing signal.

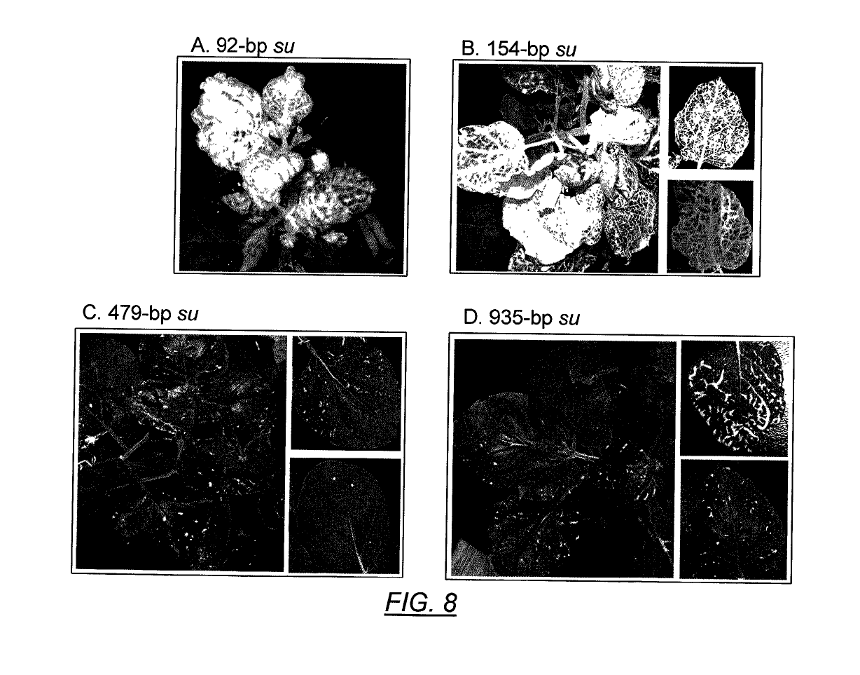

The small size requirement for the induction of silencing prompted the inventors to test various TGMV vector combinations for their ability to silence endogenous plant genes. The inventors first replaced the AR1 open reading frame with a 92-bp fragment of su. FIG. 7 (panel B) shows an example of the 92-bp su fragment in the TGMV A-derived vector, co-bombarded with wild type TGMV B, could cause silencing in vascular tissue with attenuated symptoms. The inventors then wanted to construct a vector system that would retain expression of all viral genes, and possibly increase the spread of silencing. The same 92-bp su fragment (FIG. 6) was cloned into TGMV B immediately downstream of the BR1 gene such that BR1 and foreign DNA sequences were co-transcribed from the BR1 promoter (FIG. 1, panel A). Plants inoculated with A/TGMV B::92su showed extensive silencing (FIG. 8, panel A) but developed viral symptoms that included leaf curling and stunting. In contrast, plants inoculated with a fragment containing an additional 62-bp of su (A/TGMV B::154su) showed extensive silencing and minimal symptoms (FIG. 8, panel B). These plants never outgrew silencing. This fragment was tested in both the sense and antisense orientations in the B component vector. Although systemic silencing was slightly greater in plants inoculated with the sense construct, the difference was not significant (data not shown). Southern blot analysis of total viral DNA accumulation showed that the 92-bp su fragment carried by TGMV B component supported greater TGMV replication (data not shown). To determine if sequence location was important, a 154-bp fragment from the 5′ end of the su gene was amplified by PCR and tested for silencing in the TGMV B vector. There were no significant differences in symptom development or extent of silencing between the two fragments (data not shown).

For large-scale analysis of gene function, it would be useful to clone variable-sized fragments into the B component. To test the upper limit of stable foreign DNA transmission and silencing 479-bp and 935-bp fragments of the su gene were cloned into the B component vector. These constructs supported significantly less silencing than those carrying 92-154 bp of su sequence (compare FIG. 8, panels A-D). Except for bombarded leaves, which had numerous yellow spots, extensive silencing was seen in only one of ten plants and was delayed. PCR analysis of viral DNA isolated from this plant showed that a deletion had occurred that was predicted to decrease the insert size to below 150-bp (data not shown).

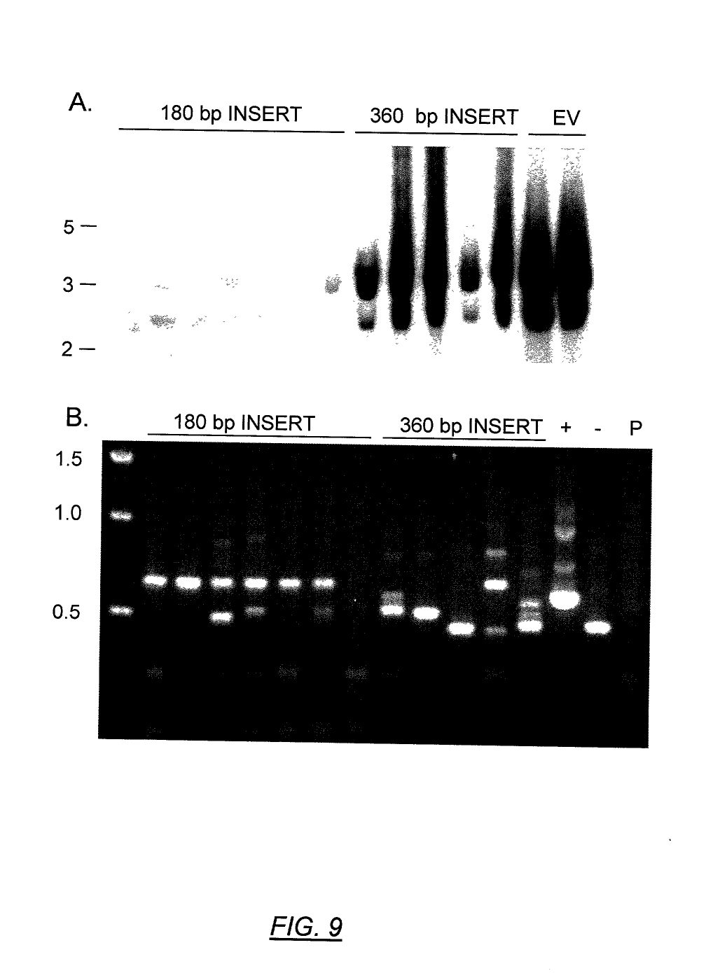

To confirm that genes other than su could be silenced using the TGMV vector system, plants were infected with wild type TGMV A and TGMV B harboring the pcna gene encoding proliferating cell nuclear antigen. Single and tandem direct repeats of a 180-bp insert homologous to the PCNA gene were subsequently tested. Plants inoculated with a single 180-bp insert lacked symptoms and showed very little DNA accumulation in new growth (data not shown). In contrast, plants inoculated with the tandem repeat showed symptoms resembling wt TGMV, and supported high levels of viral DNA replication. PCR analysis of insert size showed that while a single insert was stable, in every case the tandem repeat was deleted, allowing productive infection of upper leaves (data not shown). Sequence analysis of one of the deleted fragments revealed that fewer than 25 nt retained homology to the original PCNA insert (data not shown). This insertion of a large foreign DNA in the TGMV B vector was destabilizing. DNA was isolated from plants 4 weeks post inoculation with TGMV A/B::180PCNA or A/B::180PCNAtr, containing a tandem direct repeat of a 180-bp PCNA fragment. FIG. 9 (Panel A) shows that viral DNA accumulation in new growth of plants inoculated with a single180-bp insert was low compared to plants inoculated with the tandem repeat (360-bp insert). Accumulation of viral DNA from plants inoculated with the B component vector and wild type A was higher than the same vector with insert DNA. FIG. 9 (Panel B) shows PCR products spanning the inserted fragment from each of the plants in the upper plant. The 180-bp insert was stable whereas the tandem repeat (360-bp insert) was deleted. These results demonstrate that there is a narrow range of fragment sizes that can be stably propagated by the TGMV B vector, and that large fragments either prevent viral movement or are deleted.

CbLCV in N. Benthamiana and A. Thaliana—Insertions into the A Component.

The AR1 gene encodes the coat protein gene, which is transcribed at high levels. Removal of the AR1 gene in CbLCV allows for up to 800-bp of foreign DNA to be inserted without compromising movement. DNA fragments larger than 1 kb are not stably propagated, and only deleted forms of the CbLCV virus show systemic movement. Conversely, TGMV AR1 deletions move as circular molecules of about 1.7-kb, similar deletions in African Cassava mosaic virus only move in N. benthamiana when wild type size is restored by adding DNA, either from the ACMV A or B components (Klinkenberg et al., J. Gen. Virol. 70, 1873 (1989). ACMV has a similar genetic organization as TGMV and CbLCV. To determine if CbLCV has a strict genome size requirement for movement, the AR1 deletions were tested for movement in N. benthamiana. AR1 deletions showed systemic movement in both Arabidopsis (FIG. 10, panel A and B) and N. benthamiana (data not shown), and symptoms were attenuated as compared to wild type CbLCV (FIG. 10, panel F). Symptoms for the AR1 mutant in Arabidopsis included stunting and curling of the influorescences and leaves (FIG. 10, panels A and B). Chlorosis was not evident in the AR1 mutant.

It was investigated whether a gene, replacing the AR1 gene in CbLCV A, could silence an endogenous gene of N. benthamiana. N. benthamiana plants inoculated with a 786-bp su fragment, cloned in sense orientation, into the CbLCV A component showed yellow spots in inoculated leaves. Systemic variegation was also seen, although at markedly-reduced levels compared to TGMV vectors (data not shown). There were no apparent symptoms in new growth suggesting that viral movement may have been impaired. In one plant, silencing appeared in a sectored area, suggestive of viral DNA rearrangement, deletion, or other type of mutation.

To test the silencing of genes in A. thaliana, an 364-bp antisense Ch-42 fragment cloned into the CbLCV A component (CbLCV A::CH42) was transformed into Arabidopsis plants with fully-developed rosettes. Post-transformation, these plants did not show yellow spots and silencing was not apparent until 3 weeks later, when inflorescence stems appeared. The CbLCV A::CH42-transformed plants (FIG. 10, panels C and D) lacked chlorophyll, in contrast to stems of plants mock-inoculated (FIG. 10, panel E) and AR1 mutant-inoculated (FIG. 10, panels A and B). Siliques also lacked chlorophyll and had a strikingly uniform in yellow-white color (FIG. 10, panel D). Siliques were often bunched, perhaps to due to the virus.

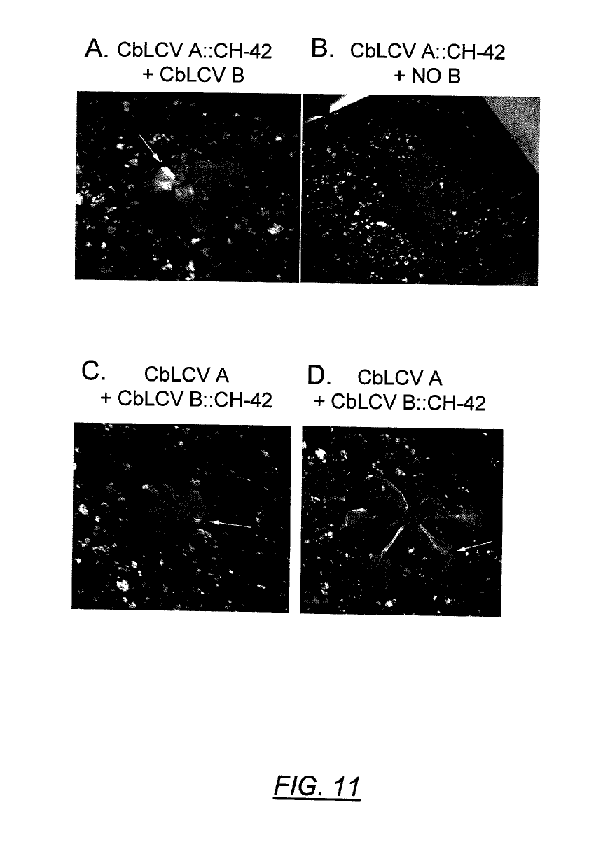

As the stage of plant development may be a factor in transformation, Arabidopsis was transformed at the 4-leaf stage with the CbLCV A::CH42 construct and a wild type B component. It was observed that silencing occurred sooner in plants at the 4-leaf stage (FIG. 11, panel A); within a week as opposed to 2.5-3 weeks for plants with rosettes and silencing was present in new growth.

CbLCV in N. Benthamiana and A. Thaliana—Insertions into the B Component.

It was investigated whether the CbLCV B component could be used as a silencing vector. A 154-bp fragment of su was cloned into the B component immediately downstream of the BR1 gene. Due to a technical oversight, the stop codon was altered and read through of the BR1 ORF into the su fragment could occur. When bombarded into N. benthamiana, this vector produced yellow spots but very little systemic silencing (data not shown). The tentative conclusion from this experiment is that the BR1 gene, which is needed for cell-to-cell and long distance movement, is also needed for systemic silencing and was disrupted when the su fragment was inserted into the vector.

To test the CbLCV B component in A. thaliana, a 144-bp fragment of Ch-42 was cloned immediately downstream of the BR1 gene. Inoculation of plants with well-developed rosettes did not produce yellow spots or significant silencing in new growth (data not shown). However, inoculation of seedlings germinated on petri plates and bombarded at the 4-leaf stage did show circular areas of chlorosis that may represent gene silencing (FIG. 11, panel B). New growth in these plants does not appear to show silencing. The plants inoculated with both A and B components did not grow well (FIG. 11, panels A, C, D) but plants inoculated with only CbLCV A::CH-42 grew well (FIG. 10, panel B). This was surprising for the CbLCV B::CH-42 plants (FIG. 11, panel B) because this vector appeared to be compromised for movement due to the insertion of Ch-42 into the open reading frame of the BR1 gene. These plants survived one week before deteriorating, suggesting that the infection and not the transplantation was the problem. It was concluded that CbLCV had a negative effect on Arabidopsis growth.

### EXAMPLE 6

### Silencing of Essential Genes

Proliferating cell nuclear antigen (PCNA) is a highly conserved processivity factor for DNA polymerase δ that is required for DNA replication and repair, and is highly expressed in dividing cells (Daidoji et al. (1992) Cell Biochem. Funct. 10:123-132; Kelman (1997) Oncogene 14:629-640). It has been previously shown that the PCNA gene is induced in mature tissues by TGMV infection (Nagar et al. (1995) Plant Cell 7:705-719; Egelkrout et al., submitted). Hence, if TGMV silenced a gene required for its own replication, a systemic TGMV infection would be prevented. To determine if TGMV could silence PCNA, plants were transformed with TGMV A::650PCNA/B. Systemic infection was significantly reduced in plants bombarded with this construct, and only 6 of 14 plants showed viral DNA accumulation by DNA gel blot analysis (data not shown).

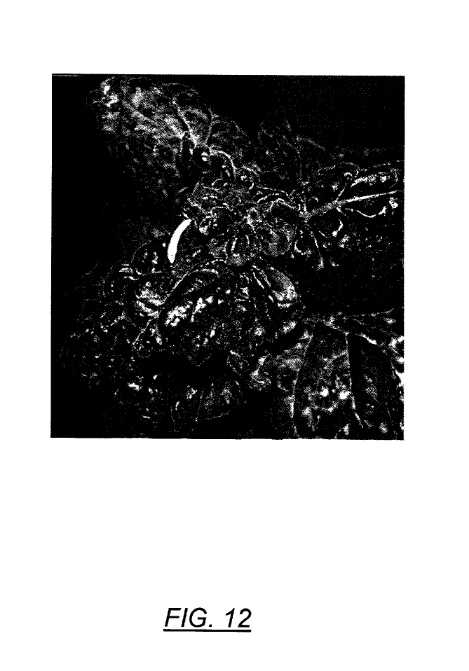

When a B component vector was used to propagate a 122-bp PCNA fragment, different results were obtained. Plants showed TGMV symptoms in lower, mature leaves but then showed greatly reduced primary growth (FIG. 12, panel A). Young leaves continued to expand, forming cabbage-like clusters at the apical meristems (FIG. 12, panel A). Leaves were often misshapen with truncated basipetal growth and little or no petiole development. These results suggested that, unlike the A vector carrying PCNA, the B vector caused silencing of PCNA expression in young tissue. This difference between the TGMV A and B vectors was not expected, but is consistent with the extensive spread of su silencing seen using the B component vector compared to A (compare FIG. 7 and FIG. 8).

### EXAMPLE 7

### A. Thaliana Ecotype Transformation

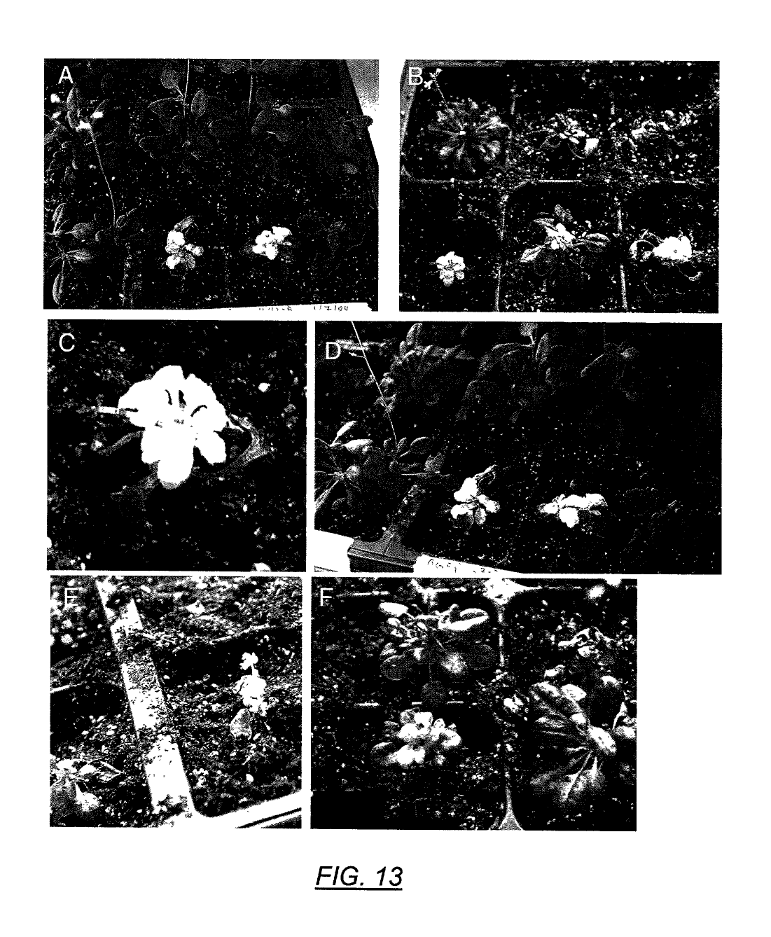

Studies were undertaken to determine whether CbLCV transformation was ecotype-specific. Ecotypes Columbia (FIG. 13, panels A-E) and Landsberg (FIG. 13, panel F), transformed at the 4-leaf stage of growth on plates with CbLCV A::CH42, demonstrated uniform silencing (yellow tissue) in plants that received DNA. The silencing appears to restrict vegetative and inflorescence development, but can be achieved in both ecotypes.

### EXAMPLE 8

### Multigene Suppression Using Geminivirus Vectors

Suppression in N. Benthamiana with TGMV.

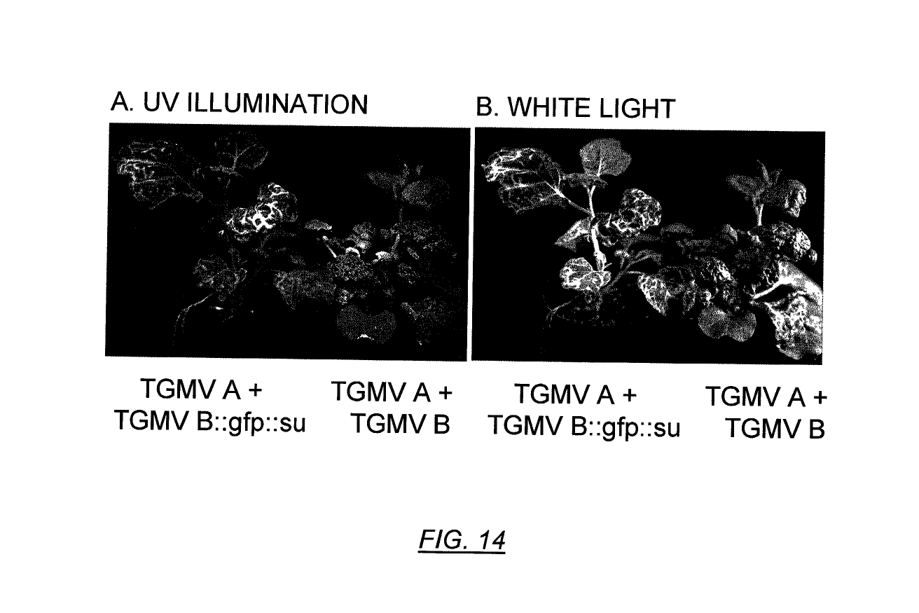

Knockout mutations may not produce a detectable phenotype if the genes have redundant functions. A rapid test for silencing combinations of genes may help to elucidate these kinds of relationships. To determine whether a single episomal silencing vector can target two different chromosomal genes, a combination of sequences designed to silence both the green fluorescent protein gene (gfp) and su were tested. A chimeric foreign DNA sequence consisting of 361 bp of su gene sequence and 388-bp of gfp gene sequence was cloned into a TGMV A vector as an AR1 gene replacement resulting in vector MT0001. Transgenic plants expressing a CaMV 35S-gfp gene were inoculated with MT0001 vector. Inoculated leaves had yellow spots, indicative of su silencing, surrounded by a larger region of gfp silencing, seen under UV illumination as a region of red chlorophyll fluorescence (data not shown). Silencing of two genes from the TGMV B vector was tested using a 140-bp chimeric DNA insert consisting of 58-bp homologous to su and 72-bp homologous to GFP. Silencing of both su and GFP was detected following bombardment into N. benthamiana carrying a CaMV 35S-GFP transgene. GFP silencing occurred throughout the plant, whereas su silencing was variable and reduced compared to GFP (FIG. 14, left plant, Panels A and B). These results demonstrate that two genes can be silenced simultaneously from the same episomal DNA construct. Similar results were obtained using a chimeric fragment inserted as an AR1 replacement in TGMV A (data not shown). In this case the fragment contained 400-bp of homology with GFP and 390-bp of homology to su. Although GFP silencing generally extended throughout the plant, su silencing was not extensive.

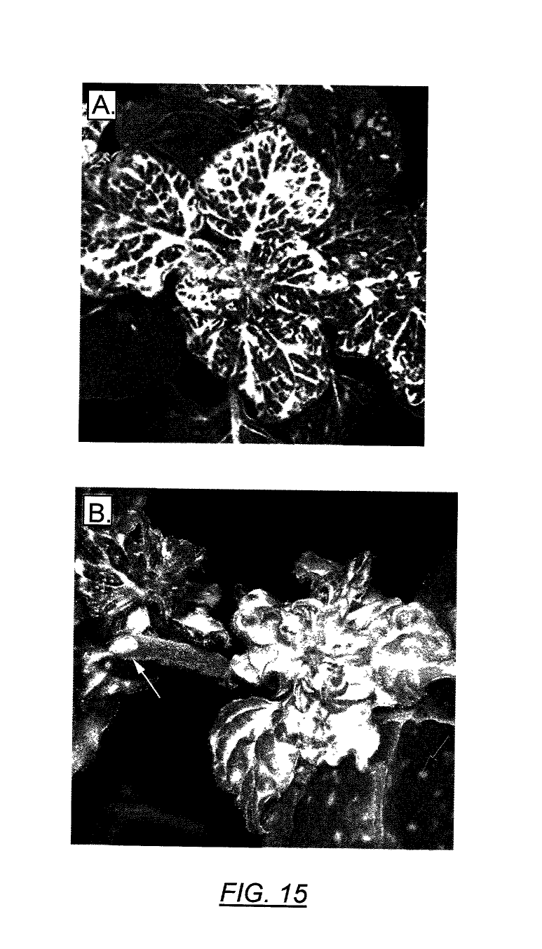

A rapid means for simultaneously silencing defined combinations of genes in intact plants would help to identify genes with redundant function. To determine whether a bipartite episomal silencing vector can target two endogenous plant genes from different components, a combination of sequences designed to silence PCNA and su, two genes essential for plant growth, were tested. An A component vector with 790-bp su was co-bombarded with a B component vector carrying a 122-bp PCNA fragment. Symptom formation from TGMV A::790su/B::122PCNA during the first 2-3 weeks resembled those of TGMV A::790su/B with yellow spots in inoculated leaves and variegated tissue in upper leaves. FIG. 15, panel A shows an example of a plant in which the apical meristem terminated primary growth, due to silencing of PCNA. Inoculated leaves showed yellow spots and remained green while upper leaves were variegated, and showed progressively reduced expansion. The terminal meristem never recovered primary growth.

Apical dominance prevents axillary bud growth in N. benthamiana. It was reasoned that release of axillary bud inhibition might allow PCNA silencing to occur from a diffusible PCNA silencing signal in adjacent, infected leaves. Inflorescence stems were pruned three weeks after inoculation with TGMV A::790su/B::122PCNA. FIG. 15, panel B shows that growth of axillary branches was severely restricted in these plants. Control plants inoculated at the same time with TGMV A::790su/B were similarly pruned but axillary branch development was normal (data not shown). Extensive su silencing also occurred in some axillary bud leaf clusters of plants inoculated with TGMV A::790su/B::122PCNA, while only variegated tissue was present in others (FIG. 15, panel B). This range of silencing phenotypes on the same plant may reflect differential movement of diffusible silencing components.

Suppression in A. Thaliana with CbLCV.

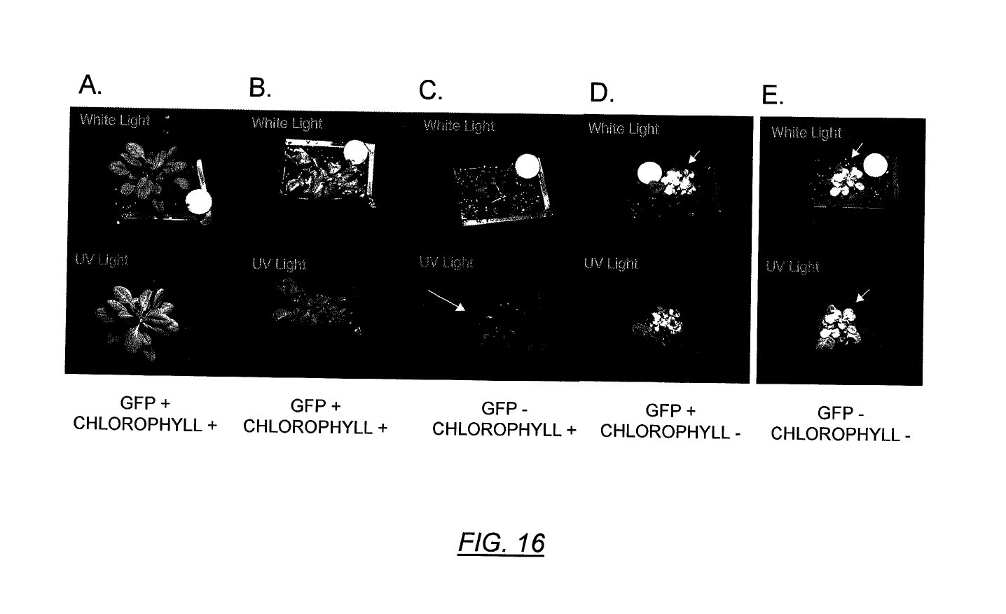

Silencing of two genes from the B component of CbLCV vector was also tested. Transgenic plants expressing a CaMV 35S-gfp gene were inoculated with the CbLCV A vector containing: (1) a coat protein deletion (-AR1), (2) a 364-bp fragment of Ch-42, (3) a 400-bp fragment of gfp, or (4) an a chimeric Ch-42::gfp fragment (CbLCV::CH42-GFP). As a control, trangenic plants were mock-inoculated to demonstrate a healthy plant (upper and lower panel, FIG. 16, panel A). Plants inoculated with CbLCV A::GFP exhibited a reduction in GFP fluorescence (lower panel, FIG. 16, panel C) compared to mock-inoculated and CbLCV A (-AR1)-inoculated plants (lower panel, FIG. 16, panels A and B) and only red autofluorescence from chlorophyll was seen (upper panel, FIG. 16, panel C). Silencing of Ch-42 was evident by yellowing of inoculated leaves (upper panel, FIG. 13, panel D) compared to no yellowing in a mock-inoculated plant (upper panel, FIG. 13, panel A). Simultaneous silencing of both gfp and Ch-42 in plants inoculated with CbLCV::CH42-GFP, was evident by a lack of chlorophyll in inoculated leaves (upper panel, FIG. 16, panel E) and a lack of GFP fluorescence (lower panel, FIG. 16, panel E). These results provide a visual demonstration that two genes can be silenced simultaneously from one DNA construct.

### EXAMPLE 9

### Systemic-Acquired Silencing and Anti-Silencing

Recently, silencing caused by diffusible factors capable of moving between cells has been reported (Palauqui et al., EMBO J. 16: 4738 (1997); Voinnet and Baulcombe, Nature 389: 553 (1997)). Evidence for a phloem-mobile component involved in post-transcriptional silencing was provided by graft transmissibility experiments (Voinnet and Baulcombe, Nature 389: 553 (1997)). These same investigators showed that a post-transcriptionally silenced transgene expressed from the CaMV 35S promoter could provide a self-perpetuating silencing factor (Anandalakshmi et al., Proc. Natl. Acad. Sci. USA. 95:13079 (1998). Grafting experiments demonstrated that while either an endogenous or a 35S promoter transgene, if silenced post-transcriptionally, could transmit a factor that spread silencing to other tissue, only the 35S promoter transgene was able to act as a source of the factor required to establish post-transcriptional gene silencing de novo.

Introduction of sequences for the green fluorescent protein (GFP) into a single leaf of a transgenic GFP plant using Agrobacterium infiltration caused silencing of the GFP gene in upper leaves remote from the site of infiltration (Palauqui et al., EMBO J. 16: 4738 (1997)). These same transgenic GFP plants were able to outgrow a cytoplasmically-localized PVX-GFP viral infection (Palauqui et al., EMBO J. 16: 4738 (1997)) perhaps because the post-transcriptional gene silencing (PTGS) machinery degraded all copies of the PVX-GFP construct. The authors concluded that gene silencing and viral resistance are functionally related. The inventors infected similar transgenic GFP N. benthamiana plants with a TGMV A component carrying the full-length gfp gene in the sense orientation, replacing the coat protein gene (TGMV A::gfp). Silencing of GFP was achieved, but the transgenic GFP plants were not cured of the TGMV A::gfp construct (data not shown). It was concluded that as geminiviruses, unlike the PVX virus, replicate in the nucleus they may be protected from the viral resistance machinery.

As a counter-measure to the plant's gene silencing/viral resistance machinery, viruses have developed anti-silencing mechanisms. Two RNA viruses have recently been shown to encode anti-silencing proteins. Tobacco etch virus encodes a P1/HC-Pro polyprotein that can reverse PTGS (Brigneti et al., EMBO J. 17:6739 (1998); Kasschau and Carrington, Cell 95: 461 (1998); Beclin et al., Virology 252: 313 (1998)). Cucumber mosaic virus contains a protein 2b that appears to inhibit the initiation of PTGS in new growth (Kasschau and Carrington, Cell 95: 461 (1998). Cucumber mosaic virus, but not tomato black ring nepovirus, prevents PTGS of an endogenous gene and a transgene (Kenton et al.,. Chromosome Res. 3: 346 (1995). These recent results provide strong evidence that gene silencing is part of the plant defense response to viral infection, and that viruses have evolved counter-defense strategies. A mutant form of the TGMV A component was identified that may separate the viral anti-silencing signal from the diffusible silencing signal. This mutant form of the A component is a Leu₁₄₈→Ala149 conversion which confers a higher level of DNA replication and possibly restricts the virus to the phloem tissue (Kong, L. J., et al, (2000) EMBO J. 19:3485-3495). When the mutant is transformed into N. benthamiana, in conjunction with a TGMV B component containing a 154-bp fragment of su (TGMV B::154su), the plants exhibit a higher degree of silencing (left plant, FIG. 5) than plants transformed with a wild type A component and TGMV B::154su (right plant, FIG. 5). It was concluded that by restricting the virus to the phloem, the diffusible silencing signal is physically-separated from the viral anti-silencing signal.

### EXAMPLE 10

### Geminivirus-Mediated Silencing in Canola

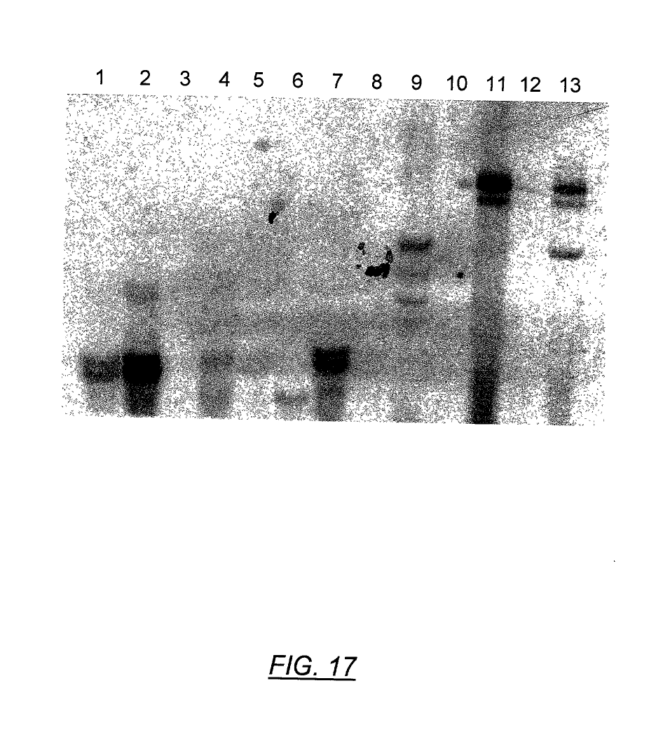

A cabbage leaf curl virus A component vector carrying a 400 bp Arabidopsis CH-42 gene fragment, which encodes a subunit of magnesium chelatase required for chlorophyll formation, were used to inoculate canola (Brassica napus). The CH-42 sequence replaced part of the coding sequence for the CbLCV AR1 protein. Canola is in the same family as Arabidopsis and the same genus as cabbage. Compared with Arabidopsis, limited evidence of silencing (FIG. 17) was observed but the extent of homology between the 400 bp Arabidopsis CH-42 gene and the B. napus CH-42 gene is undetermined, and may not have been high enough to induce silencing. Arrows in FIG. 17 indicate mild symptoms.

DNA gel blots probed with cabbage leaf curl virus showed that input DNA from microprojectile bombardment remained on the surface of the leaves. Replication of the silencing vector was demonstrated by digesting with DpnI, an enzyme that digests DNA made in E. coli, but not in plants. The smaller bands seen in FIG. 17B show the vector after it has replicated. The larger bands in FIG. 17B show the vector carried by input plasmid DNA. Lane 8 is the control and contains canola DNA that was mock-bombarded.

These results indicate that a CbLCV silencing vector can replicate in canola. Moreover, no pathogenicity was observed in inoculated canola plants.

Canola plants are modified to stably integrate a green fluorescent protein (gfp) transgene. A CbLCV A component silencing vector is constructed in which a portion of the AR1 coding sequence is replaced with a fragment of the gfp gene in the sense or antisense direction. This CbLCV A::gfp vector is inoculated into the transgenic canola plants stably expressing the gfp transgene. The plants are allowed to grow for a period of time and are then observed for gfp silencing.

The foregoing examples are illustrative of the present invention, and are not to be construed as limiting thereof. The invention is described by the following claims, with equivalents of the claims to be included therein.

That which is claimed is:

1. A cabbage leaf curl virus (CbLCV) silencing vector comprising a CbLCV genomic component comprising one or more heterologous DNA sequences, each of the heterologous DNA sequences having substantial sequence similarity to an endogenous plant gene.

2. The silencing vector of claim 1, wherein the silencing vector comprises the CbLCV A component.

3. The silencing vector of claim 2, wherein the silencing vector further comprises geminivirus BR1 and BL1 genes.

4. The silencing vector of claim 3, wherein the geminivirus BR1 and BL1 genes are from CbLCV.

5. The silencing vector of claim 2, wherein at least one of the heterologous DNA sequences is inserted into or replaces a segment of the coding sequence for the CbLCV coat protein.

6. The silencing vector of claim 5, wherein at least one of the heterologous DNA sequences replaces a segment of the coding sequence for the CbLCV coat protein.

7. The silencing vector of claim 2, wherein at least one of the heterologous DNA sequences is inserted into or replaces a segment of the common region.

8. The silencing vector of claim 2, wherein each of the heterologous DNA sequences is at least about 20 base pairs in length, and further wherein the combined length of the heterologous DNA sequences does not exceed about 800 base pairs in length.

9. The silencing vector of claim 1, wherein the silencing vector comprises the CbLCV B component.

10. The silencing vector of claim 9, wherein the silencing vector further comprises the geminivirus AL1 and AL3 genes.

11. The silencing vector of claim 10, wherein the geminivirus AL1 and AL3 genes are from CbLCV.

12. The silencing vector of claim 10, wherein the silencing vector further comprises the geminivirus AL2 gene.

13. The silencing vector of claim 9, wherein each of the heterologous DNA sequences is at least about 20 base pairs in length, and further wherein the combined length of the heterologous DNA sequences does not exceed about 200 base pairs in length.

14. The silencing vector of claim 9, wherein at least one of the heterologous DNA sequences is inserted into or replaces a segment following the stop codon of the BR1 gene.

15. The silencing vector of claim 9, wherein at least one of the heterologous DNA sequences is inserted into or replaces a segment following the stop codon of the BL1 gene.

16. The silencing vector of claim 9, wherein at least one of the heterologous DNA sequences is inserted into or replaces a segment of the intergenic region .

17. The silencing vector of claim 9, wherein at least one of the heterologous DNA sequences is inserted into or replaces a segment of the common region.

18. The silencing vector of claim 9, wherein at least one of the heterologous DNA sequences is inserted into or replaces a segment of the coding region of the BL1 or BR1 genes.

19. The silencing vector of claim 9, wherein at least one of the heterologous DNA sequence is inserted into the 3′ untranslated sequence of the BR1or BL1 genes.

20. The silencing vector of claim 1, wherein expression of the one or more heterologous DNA sequences modifies one or more observable plant phenotypic traits.

21. The silencing vector of claim 1, wherein the silencing vector comprises two or more heterologous DNA sequences having substantial sequence similarity to an endogenous plant gene.

22. The silencing vector of claim 21, wherein the two or more heterologous DNA sequences have substantial sequence similarity to two or more non-homologous endogenous plant genes.

23. The silencing vector of claim 21, wherein the two or more heterologous DNA sequences have substantial sequence similarity with two or more genes within a biochemical pathway.

24. The silencing vector of claim 1, wherein the at least one heterologous DNA sequence has at least 85% sequence identity to an endogenous plant gene.

25. The silencing vector of claim 1, further comprising a heterologous DNA sequence having substantial sequence similarity to a gene encoding a reporter protein.

26. The silencing vector of claim 1, wherein at least one of the heterologous DNA sequences has substantial sequence similarity to a gene encoding a non-translated RNA molecule.

27. The silencing vector of claim 1, wherein each of the heterologous DNA sequences is operably associated with a promoter.

28. The silencing vector of claim 27, wherein the heterologous DNA sequences are operably associated with a single promoter.

29. The silencing vector of claim 28, wherein the promoter is the CbLCV coat protein promoter.

30. The silencing vector of claim 1, wherein at least one of the heterologous DNA sequences is in the sense orientation.

31. The silencing vector of claim 1, wherein at least one of the heterologous DNA sequences is in an antisense orientation.

32. The silencing vector of claim 1, wherein at least one of the heterologous DNA sequences has substantial sequence similarity to a fragment of an endogenous plant gene.

33. The silencing vector of claim 32, wherein at least one of the heterologous DNA sequences has substantial sequence similarity to the coding region of an endogenous plant gene.

34. The silencing vector of claim 1, wherein at least one of the heterologous DNA sequences has substantial sequence similarity to an endogenous plant promoter sequene.

35. The silencing vector of claim 1, wherein the silencing vector is a shuttle vector that replicates in a non-plant cell.

36. The shuttle vector of claim 35, wherein the shuttle vector replicates in a bacterial cell.

37. The shuttle vector of claim 36, wherein the shuttle vector is a plasmid.

38. A silencing vector comprising a Cabbage Leaf Curl Virus (CbLCV) origin of replication, CbLCV sequences encoding proteins sufficient for replication of said silencing vector in a plant cell, and one or more heterologous DNA sequences, each of the heterologous DNA sequences having substantial sequence similarity to an endogenous plant gene.

39. The silencing vector of claim 38, wherein the silencing vector comprises a CbLCV common region.

40. The silencing vector of claim 38, wherein the silencing vector comprises a geminivirus AL3 gene.

41. The silencing vector of claim 38, wherein the silencing vector comprises a geminivirus AL1 gene.

42. The silencing vector of claim 38, wherein the silencing vector comprises a geminivirus AL2 gene.

43. The silencing vector of claim 38, wherein the silencing vector comprises geminivirus BR1 and BL1 genes.

44. The silencing vector of claim 38, wherein the silencing vector comprises the common region from the CbLCV A component, a geminivirus AL1 gene, and a geminivirus AL3 gene.

45. The silencing vector of claim 38, wherein the silencing vector comprises a CbLCV coat protein promoter operably associated with at least one of the one or more heterologous DNA sequences.

46. The silencing vector of claim 38, wherein the at least one heterologous DNA sequence has at least 85% sequence identity to an endogenous plant gene.

47. The silencing vector of claim 38, wherein at least one of the heterologous DNA sequences is inserted into the silencing vector outside of the geminivirus sequences.

48. The silencing vector of claim 38, wherein expression of the one or more heterologous DNA sequences modifies one or more observable plant phenotypic traits.

49. The silencing vector of claim 38, wherein the one or more heterologous DNA sequences has substantial sequence similarity to two or more endogenous plant genes.

50. The silencing vector of claim 49, wherein the one or more heterologous DNA sequences has substantial sequence similarity to two or more non-homologous endogenous plant genes.

51. The silencing vector of claim 38, wherein at least one of the heterologous DNA sequences has substantial sequence similarity to a fragment of an endogenous plant gene.

52. The silencing vector of claim 51, wherein at least one of the heterologous DNA sequences has substantial sequence similarity to the coding region of an endogenous plant gene.

53. The silencing vector of claim 38, wherein at least one of the heterologous DNA sequences has substantial sequence similarity to an endogenous plant promoter sequence.

54. The silencing vector of claim 38, wherein the silencing vector is a shuttle vector that replicates in a non-plant cell.

55. The shuttle vector of claim 54, wherein the shuttle vector replicates in a bacterial cell.

56. The shuttle vector of claim 55, wherein the shuttle vector is a plasmid.

57. A silencing vector comprising a Cabbage Leaf Curl Virus (CbLCV) origin of replication, a CbLCV BR1 or BL1 gene, and one or more heterologous DNA sequences, each of the heterologous DNA sequences having substantial sequence similarity to an endogenous plant gene.

58. The silencing vector of claim 57, wherein the silencing vector comprises both the CbLCV BR1 and BL1 genes.

59. The silencing vector of claim 58, wherein the one or more heterologous DNA sequences are inserted into or replace a segment of one or more of:
(a) the region downstream from the stop codon of the BR1 gene;
(b) the region downstream from the stop codon of the BL1 gene;
(c) the coding region of the BR1 gene; and
(d) the coding region of the BL1 gene.

60. The silencing vector of claim 58, wherein the silencing vector further comprises DNA sequences encoding proteins sufficient to support the replication of the silencing vector in a plant cell.

61. The silencing vector of claim 60, wherein the DNA sequences encoding the replication proteins are CbLCV sequences.

62. A plant cell comprising the silencing vector of claim 1.

63. A plant comprising the plant cell of claim 62.

64. A plant cell comprising the silencing vector of claim 38.

65. A plant comprising the plant cell of claim 64.

66. A plant cell comprising the silencing vector of claim 57.

67. A plant comprising the plant cell of claim 66.

68. An Arabidopsis cell comprising the silencing vector of claim 1.

69. An Arabidopsis plant comprising the cell of claim 68.

70. A method of silencing the expression of one or more endogenous plant genes, comprising inoculating a plant cell with the silencing vector of claim 1.

71. The method of claim 70, wherein the plant cell is from a species of Brassicaceae.

72. The method of claim 71, wherein the plant cell is an Arabidopsis cell.

73. The method of claim 71, wherein the plant cell is a canola cell.

74. The method of claim 70, wherein the plant cell is a tobacco cell.

75. The method of claim 70, wherein the plant cell is selected from the group consisting of a mesophyll cell, epidermis cell, cortical cell, parenchymal cell, guard cell, xylem cell, floral cell, fruit cell, seed coat cell, meristematic cell, apical cell, sclerenchyma cell, and colenchyma cell.

76. The method of claim 70, wherein the silencing vector comprises the CbLCV A component.

77. The method of claim 76, further comprising inoculating the plant cell with an additional vector comprising a CbLCV B component.

78. The method of claim 77, wherein the additional vector is a silencing vector.

79. The method of claim 76, wherein the plant cell is stably transformed with and expresses the CbLCV BR1 and BL1 genes.

80. The method of claim 70, wherein the silencing vector comprises the CbLCV B component.

81. The method of claim 81, wherein the plant cell is stably transformed with and expresses the CbLCV AL1, AL2 and AL3 genes.

82. A method of silencing the expression of one or more plant genes, comprising inoculating a plant cell with the silencing vector of claim 38.

83. The method of claim 82, wherein the plant cell is from a species of Brassicaceae.

84. A method of silencing the expression of one or more plant genes, comprising inoculating a plant cell with the silencing vector of claim 57.

85. The method of claim 84, wherein the plant cell is from a species of Brassicaceae.

86. A method of silencing expression of one or more endogenous plant genes, comprising inoculating a plant with the silencing vector of claim 1.

87. The method of claim 86, wherein expression of the one or more plant genes is systemically silenced in the plant.

88. A method of silencing expression of one or more endogenous plant genes, comprising inoculating a plant with the silencing vector of claim 38.

89. The method of claim 88, wherein expression of the one or more plant genes is systemically silenced in the plant.

90. A method of silencing expression of one or more endogenous plant genes, comprising inoculating a plant with the silencing vector of claim 57.

91. The method of claim 90, wherein expression of the one or more plant genes is systemically silenced in the plant.

92. A method of screening an isolated plant DNA sequence for function, comprising:
inoculating a plant with a silencing vector according to claim 1, wherein at least one of the heterologous DNA sequences has substantial sequence similarity to the isolated plant DNA sequence; and
comparing the inoculated plant to control plant tissue;
wherein differences between the inoculated and control plant tissues indicate the function of the isolated plant DNA sequence.

93. The method of claim 92, wherein the inoculated plant comprises the control plant.

94. A method of screening for the function of one or more endogenous plant genes, comprising:
inoculating a plant with a silencing vector according to claim 1, wherein at least one of the heterologous DNA sequences has substantial sequence similarity to an endogenous plant gene; and
comparing the inoculated plant with control plant tissue;
wherein differences between the inoculated and control plant tissues indicate the function of the one or more plant genes.

95. The method of claim 94, wherein the inoculated plant comprises the control plant tissue.


SEQUENCE LISTING (7 sequences)

SEQ ID NO: 1 - DNA, 28 bp, Artificial Sequence
  misc_feature (1)..(28): Synthetic Oligonucleotide.
gatctagagggaggaagttttatggagg

SEQ ID NO: 2 - DNA, 29 bp, Artificial Sequence
  misc_feature (1)..(29): Synthetic Oligonucleotide.
gatctagatagctgcaaatggatacaccg

SEQ ID NO: 3 - DNA, 24 bp, Artificial Sequence
  misc_feature (1)..(24): Synthetic Oligonucleotide.
actgttagatctttagttgatctg

SEQ ID NO: 4 - DNA, 30 bp, Artificial Sequence
  misc_feature (1)..(30): Synthetic Oligonucleotide.
aatcccttctctagaaaccgtaatccaacc

SEQ ID NO: 5 - DNA, 10 bp, Artificial Sequence
  misc_feature (1)..(10): Synthetic Oligonucleotide Linker.
aaggtacctt

SEQ ID NO: 6 - DNA, 20 bp, Artificial Sequence
  misc_feature (1)..(20): Synthetic Oligonucleotide.
gtcggatattgtgtcaaagg

SEQ ID NO: 7 - DNA, 20 bp, Artificial Sequence
  misc_feature (1)..(20): Synthetic Oligonucleotide.
tctactattgggctaacagg